(19) United States
(12) Patent Application Publication
(10) Pub. No.: US 2025/0169989 A1
(43) Pub. Date: May 29, 2025

(54) METHODS AND DEVICES FOR INCREASING AQUEOUS DRAINAGE OF THE EYE

(71) Applicant: Iantrek, Inc., Harrison, NY (US)

(72) Inventors: David Robson, Harrison, NY (US); Roderick H. Beaulieu, Harrison, NY (US); Sean Buxton, Harrison, NY (US); Tsontcho Ianchulev, Harrison, NY (US); Daniel Nelsen, Harrison, NY (US)

(21) Appl. No.: 18/957,401

(22) Filed: Nov. 22, 2024

Related U.S. Application Data
(60) Provisional application No. 63/558,464, filed on Feb. 27, 2024.
(60) Provisional application No. 63/604,016, filed on Nov. 29, 2023.

(51) Int. Cl. A61F 9/007; A61B 17/32; A61M 25/00

(52) U.S. Cl. CPC A61F 9/00781; A61B 17/320016; A61M 25/0068; A61M 2025/0096

(57) ABSTRACT

A device for disrupting tissue in an eye configured for ab interno insertion. An elongate, flexible shaft having a distal end region with a first tissue disruptor formed on an inward surface of the distal end region and a second tissue disruptor formed on an outward surface of the distal end region. The distal-most end is configured to be inserted into a portion of Schlemm's Canal and advanced along a circumferential contour of Schlemm's Canal away from the portion of Schlemm's Canal. The first tissue disruptor is configured to disrupt trabecular meshwork tissue as the shaft advances along the circumferential contour and the second tissue disruptor is configured to disrupt tissue upon retraction of the shaft and not as the shaft is advanced along the circumferential contour. Related methods and devices are provided.

### CROSS-REFERENCE TO RELATED APPLICATIONS

This application claims the benefit of priority to U.S. Provisional Application Ser. No. 63/604,016, filed Nov. 29, 2023, and 63/558,464, filed Feb. 27, 2024. The entire contents of these applications are incorporated by reference in their entireties.

### BACKGROUND

Current trabecular excision devices typically use excisional blades or sharp needles (e.g., goniotomy). These devices typically create single stab-like partial cuts of the trabecular meshwork. More recent devices, such as the Kahook dual blade (U.S. Pat. No. 9,872,799), Baerveldt (U.S. Pat. No. 9,999,544) and the cauterizing/plasma cutting blades of the Trabectome (U.S. Pat. No. 9,820,885), all have a sharp incisional or ablative cutting surface for use on the trabecular meshwork. As such, they all suffer from the major clinical disadvantage related to the sharp cutting nature in the process of meshwork engagement. The sharp blades often create interrupted, discontinuous, and incongruous cuts of the trabecular meshwork, which are imprecise and more akin to tissue maceration rather than the desired tissue extraction with non-lacerating atraumatic removal. This is also often associated with significant bleeding and collateral damage of both sclera, endothelium, and iris tissue. Furthermore, a single cutting blade may simply open the trabecular meshwork without removing much material. To remove material, some prior art devices provide two spaced-apart cutting elements (side-by-side) in an attempt to remove meshwork material between the cutting elements.

### SUMMARY

In an aspect, disclosed is a device for disrupting tissue in an eye. The device includes a distal portion sized and configured for ab interno insertion into an anterior chamber of the eye. The distal portion includes an elongate, flexible shaft having a distal end region; a distal-most end; a first tissue disruptor proximal of the distal-most end formed on an inward surface of the distal end region; and a second tissue disruptor proximal of the distal-most end formed on an outward surface of the distal end region. During use, the distal-most end is configured to be inserted through trabecular meshwork tissue and into a portion of Schlemm's Canal and the shaft is configured to be advanced along a circumferential contour of Schlemm's Canal away from the portion of Schlemm's Canal. The first tissue disruptor is configured to disrupt trabecular meshwork tissue as the shaft advances along the circumferential contour of Schlemm's Canal, and the second tissue disruptor is configured to disrupt tissue upon retraction of the shaft and not as the shaft is advanced along the circumferential contour.

The distal-most end can be an atraumatic tip. The atraumatic tip can be configured for circumferential gonio-traction. The atraumatic tip on the shaft can be located 1 mm-3 mm away from the first tissue disruptor and the second tissue disruptor. The shaft can be a tube having a lumen extending along a longitudinal axis and defined by a cylindrical wall. The first tissue disruptor can be a segment of the cylindrical wall of the tube projecting inward at an angle relative to the longitudinal axis. The second tissue disruptor can be a discontinuity in the cylindrical wall of the tube. The discontinuity can form a window having a leading edge facing proximally and a trailing edge facing distally. The trailing edge can be blunt so it does not disrupt tissue during advancement of the shaft, and the leading edge can be sharpened so it disrupts tissue during retraction of the shaft. The device can optionally include an inner member having a control wire and an atraumatic distal tip, the inner member being movable through the lumen of the tube. The atraumatic distal tip can be configured to be positioned distal to the distal-most end of the shaft.

The radially inward surface of the distal end region can be connected to the radially outward surface by two lateral sides. The first tissue disruptor can have a distal face, a proximal face, and a maximum thickness, the distal face projecting a distance from a first thickness of the shaft distal to the first tissue disruptor forming the maximum thickness and the proximal face tapering down from the maximum thickness to a second thickness of the shaft proximal to the tissue disruptor, and wherein the first tissue disruptor is a blunt tissue-engaging surface without any cutting element. The first thickness of the shaft between the inward and outward surfaces proximal to the disruptor can be 100-150 microns and the second thickness of the shaft between the inward and outward surfaces distal to the disruptor can be 100-150 microns. The maximum thickness of the tissue disruptor between the inward and the outward surfaces can be about 250-600 microns. The first thickness of the shaft between the inward and outward surfaces proximal to the disruptor can be 100-2000 microns and the second thickness of the shaft between the inward and outward surfaces distal to the disruptor can be 100-550 microns. The maximum thickness of the tissue disruptor between the inward and outward surfaces can be about 450-600 microns. The shaft can have a cross-sectional shape taken transverse to a length of the shaft between that is non-circular. The cross-sectional shape can be square or rectangular. The shaft can be formed of super-elastic memory-shape material. The super-elastic memory-shape material can be Nitinol. The shaft can be cut from a flat sheet of material having a thickness of about 75-550 microns to form a profile of the first and second tissue disruptors. The shaft can be formed of a material comprising Nitinol, stainless steel, or a polymer.

The second tissue disruptor can include one or more tines having a leading surface facing distally and a trailing surface facing proximally. The leading surface facing distally can be smooth to slide along an outer wall of Schlemm's Canal during advancement without causing tissue disruption. The trailing surface facing proximally can be sharp to catch on the outer wall of Schlemm's Canal during retraction causing tissue disruption. The device further includes a proximal portion that is configured to remain outside the eye when the distal portion is inserted inside the eye. The proximal portion can include an actuator operatively coupled to the shaft, the actuator configured to advance the shaft distally. The distal end region can be straight or shaped into a curve having a central plane. The curve of the distal end region of the shaft can have a radial curvature of 5-20 mm.

The device can optionally include a proximal housing having an introducer tube projecting from a distal end region of the housing, at least a portion of the shaft extending through a lumen of the introducer tube. The shaft can be configured to be advanced from the introducer tube. The shaft can develop a spring-load as the shaft extends from the introducer tube. The shaft can apply a radially outward force as the shaft extends from the introducer tube. A stiffness of the shaft can be varied by changing a length of the shaft extending from the introducer tube. The introducer tube can be a substantially rigid tube having a proximal end region that extends away from the proximal housing along a longitudinal axis and a distal end region that curves relative to the longitudinal axis.

In an interrelated aspect, provided is a device for disrupting tissue in an eye including a distal portion sized and configured for ab interno insertion into an anterior chamber of the eye having an elongate, flexible shaft including a distal end region having an inward surface, an outward surface, and a first thickness between the inward surface and the outward surface. A probe tip is at a distal-most end of the distal end region, the probe tip having a maximum thickness between the radially inward surface of the distal end region and the radially outward surface of the distal end region. A tissue disruptor is proximal of the probe tip projecting away from the radially inward surface. A neck region is proximal of the probe tip and distal to the tissue disruptor. The neck region has a second thickness between the radially inward surface and the radially outward surface. During use, the distal-most end is configured to be inserted through trabecular meshwork tissue and into a portion of Schlemm's Canal and the shaft is configured to be advanced along a circumferential contour of Schlemm's Canal away from the portion of Schlemm's Canal. The tissue disruptor is configured to disrupt trabecular meshwork tissue as the shaft advances along the circumferential contour of Schlemm's Canal. Maximum thickness of the probe tip is greater than the first thickness of the distal end region of the shaft, and the second thickness of the neck region is less than the first thickness.

The probe tip can be located 1 mm-3 mm away from the tissue disruptor. The radially inward surface of the distal end region can be connected to the radially outward surface by two lateral sides. The shaft can be formed of a super-elastic memory-shape material. The super-elastic memory-shape material can be Nitinol. The distal end region can be straight or shaped into a curve having a central plane. The curve of the distal end region of the shaft can have a radial curvature of 5-20 mm. The first thickness of the distal end region of the shaft can be at least 120 microns up to about 150 microns. The maximum thickness of the probe tip can be greater than 180 microns up to about 360 microns. The second thickness of the neck region can be less than about 100 microns down to about 60 microns.

Any of the devices described herein can be used to perform ab interno continuous goniotomy and inner wall trabeculotomy along a segment of a circumference of an eye. The segment can be greater than 90 degrees up to about 180 degrees. The method of using the device can optionally include positioning at least one implant within a ciliary cleft. The positioning can be performed after the continuous goniotomy and an inner wall trabeculotomy. The implant can be minimally-modified biological tissue. The biological tissue can be scleral, corneal, or amniotic membrane tissue.

In an interrelated aspect, provided is a device for disrupting tissue in an eye having a distal portion sized and configured for ab interno insertion into an anterior chamber of the eye having an elongate, flexible shaft with a tissue disruptor proximal of a distal-most end of the shaft formed on an inward surface of a distal end region of the shaft. During use, the shaft is configured to be advanced along a circumferential contour of Schlemm's Canal and the tissue disruptor is configured to disrupt trabecular meshwork tissue during advancement.

The tissue disruptor can be configured to disrupt trabecular meshwork tissue as the shaft advances along the circumferential contour of Schlemm's Canal. The device can optionally have a second tissue disruptor proximal of the distal-most end formed on an outward surface of the distal end region. The second tissue disruptor can be configured to disrupt an outer wall of Schlemm's Canal as the tissue disruptor is advanced along the circumferential contour. The second tissue disruptor can be configured to disrupt tissue upon retraction of the shaft and not as the shaft is advanced along the circumferential contour. The device can optionally include a proximal housing having an introducer tube coupled to a distal end region of the housing. At least a portion of the shaft can extend through a lumen of the introducer tube and configured to be advanced through the lumen the introducer tube. The introducer tube can be a substantially rigid tube having a proximal end region that extends away from the proximal housing along a longitudinal axis and a distal end region that curves relative to the longitudinal axis. The distal end region of the introducer tube can be straight or can curve forming an angle relative to the longitudinal axis. The angle can be about 90 degrees up to about 120 degrees. The distal end region of the introducer tube can curve forming an angle relative to the longitudinal axis that is about 105 degrees. The shaft can extend from the distal end region of the introducer tube forming an angle of extension relative to an axis perpendicular to the longitudinal axis that is less than about 45 degrees, or about 10-40 degrees. The introducer tube can optionally include a beveled tip forming a distal opening from the lumen of the introducer tube. The beveled tip can have a first region forming a first curve and a second region forming a second curve with a transition point between the first region and the second region. The first curve can have a radius of curvature that is larger than a radius of curvature of the second curve. The device can optionally include a stiffening sleeve fixed to an external surface of the shaft along at least a proximal region of the shaft. The stiffening sleeve can have a stiffness that functions to reinforce at least the proximal region of the shaft as the distal end region of the shaft extends through eye tissue. The shaft can have a first thickness between the radially inward surface and a radially outward surface of the distal end region. The device can optionally include a probe tip at the distal-most end of the shaft. The probe tip can have a maximum thickness between the radially inward surface and the radially outward surface. The tissue disruptor can be located proximal of the probe tip. A neck region can be proximal of the probe tip and distal to the tissue disruptor. The neck region can have a second thickness between the radially inward surface and the radially outward surface. The maximum thickness of the probe tip can be greater than the first thickness of the distal end region of the shaft, and the second thickness of the neck region can be less than the first thickness. The probe tip can be located 1 mm-3 mm away from the tissue disruptor. The first thickness of the distal end region of the shaft can be about 100 microns up to about 150 microns. The maximum thickness of the probe tip can be greater than about 180 microns and can be less than about 360 microns. The second thickness of the neck region can be less than about 100 microns down to about 50 microns. The probe tip can be an enlarged feature that is bulbous in shape. The shaft can be straight from a proximal end region to a distal end region of the shaft. At least the distal end region of the shaft can be sufficiently flexible to substantially conform to a curvature of Schlemm's Canal as the shaft is advanced along the circumferential contour of Schlemm's Canal and engages with an outer wall of Schlemm's canal. At least a distal end region of the shaft can be shape-set to define a plane of curvature so as to substantially conform to a curvature of Schlemm's Canal as the shaft is advanced along the circumferential contour of Schlemm's Canal and engages with an outer wall of Schlemm's canal. The tissue disruptor can optionally include one or more flex regions configured to move the tissue disruptor from a collapsed, smaller outer dimension configuration to an expanded, larger outer dimension configuration and return from the expanded, larger outer dimension configuration to the collapsed, smaller outer dimension configuration.

In an interrelated aspect, provided is a device for disrupting tissue in an eye having a distal portion sized and configured for ab interno insertion into an anterior chamber of the eye having an elongate, flexible shaft with a tissue disruptor proximal of a distal-most end of the shaft formed on an outward surface of a distal end region of the shaft. During use, the shaft is configured to be advanced along a circumferential contour of Schlemm's Canal. The tissue disruptor is configured to disrupt an outer wall of Schlemm's Canal.

The tissue disruptor can be configured to disrupt the outer wall as the tissue disruptor is advanced along the circumferential contour of Schlemm's Canal. The tissue disruptor can be configured to disrupt the outer wall of Schlemm's Canal upon retraction of the shaft and not as the shaft is advanced along the circumferential contour. The device can include a proximal housing having an introducer tube coupled to a distal end region of the housing. At least a portion of the shaft extends through a lumen of the introducer tube and is configured to be advanced through the lumen the introducer tube. The introducer tube can be a substantially rigid tube having a proximal end region that extends away from the proximal housing along a longitudinal axis and a distal end region that curves relative to the longitudinal axis. The distal end region of the introducer tube can curve forming an angle relative to the longitudinal axis that is about 90 degrees up to about 120 degrees. The distal end region of the introducer tube can curve forming an angle relative to the longitudinal axis that is about 105 degrees. The shaft can extend from the distal end region of the introducer tube forming an angle of extension relative to an axis perpendicular to the longitudinal axis that is less than about 45 degrees, or about 10-40 degrees. The introducer tube can optionally include a beveled tip forming a distal opening from the lumen of the introducer tube. The beveled tip can have a first region forming a first curve and a second region forming a second curve with a transition point between the first region and the second region. The first curve can have a radius of curvature that is larger than a radius of curvature of the second curve. The device can optionally include a stiffening sleeve fixed to an external surface of the shaft along at least a proximal region of the shaft. The stiffening sleeve can have a stiffness that functions to reinforce at least the proximal region of the shaft as the distal end region of the shaft extends through eye tissue. T shaft can have a first thickness between the radially inward surface and a radially outward surface of the distal end region. A probe tip can be at the distal-most end of the shaft and have a maximum thickness between the radially inward surface and the radially outward surface. The tissue disruptor can be located proximal of the probe tip. A neck region can be proximal of the probe tip and distal to the tissue disruptor. The neck region can have a second thickness between the radially inward surface and the radially outward surface. The maximum thickness of the probe tip can be greater than the first thickness of the distal end region of the shaft, and the second thickness of the neck region can be less than the first thickness. The probe tip can be located 1 mm-3 mm away from the tissue disruptor. The first thickness of the distal end region of the shaft can be about 100 microns up to about 150 microns. The maximum thickness of the probe tip can be greater than about 180 microns and less than about 360 microns. The second thickness of the neck region can be less than about 100 microns down to about 50 microns. The probe tip can be an enlarged feature that is bulbous in shape. The shaft can be straight from a proximal end region to a distal end region of the shaft. At least the distal end region of the straight shaft is sufficiently flexible to substantially conform to a curvature of Schlemm's Canal as the shaft is advanced along the circumferential contour of Schlemm's Canal and engages with an outer wall of Schlemm's canal. At least a distal end region of the shaft can be shape-set to define a plane of curvature so as to substantially conform to a curvature of Schlemm's Canal as the shaft is advanced along the circumferential contour of Schlemm's Canal and engages with an outer wall of Schlemm's canal. The tissue disruptor can optionally include one or more flex regions configured to move the tissue disruptor from a collapsed, smaller outer dimension configuration to an expanded, larger outer dimension configuration and return from the expanded, larger outer dimension configuration to the collapsed, smaller outer dimension configuration. The distal portion can be configured to maintain trabecular meshwork tissue substantially intact as the shaft is advanced along a circumferential contour of Schlemm's Canal and/or as the shaft is withdrawn along the circumferential contour of Schlemm's Canal.

Any of the devices described herein can optionally be part of a system including an implant configured to be positioned within at least a region of an eye for aqueous outflow. The implant can be minimally-modified biological tissue. The biological tissue can be scleral, corneal, or amniotic membrane tissue.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are not to scale in absolute terms or comparatively but are intended to be illustrative. Also, relative placement of features and elements may be modified for the purpose of illustrative clarity.

FIG. 1A shows an implementation of a device for removing tissue from an eye having a housing with an actuator to manipulate a tissue engager relative to an introducer tube;

FIG. 1B shows the device of FIG. 1A with the disruptor wire retracted;

FIG. 1C is a detail view of a distal end region of the device of FIG. 1A;

FIG. 1D is a detail view of an alternative distal end region of an introducer tube;

FIG. 2A is a cross-sectional partial view of a distal end region of an implementation of a device for removing tissue from an eye;

FIG. 2B is a side view of a shaft for use with the device of FIG. 2A illustrating a straight shape setting of a cut tube;

FIG. 2C is a side view of a shaft for use with the device of FIG. 2A illustrating a curved shape-setting of a cut tube;

FIG. 2D is a detail side view of the distal end region of the shaft of FIG. 2C showing the disruptor configuration;

FIG. 2E is a detail distal end perspective view of the radially inward surfaces of the distal end region of the shaft shown in FIG. 2D;

FIG. 2F is a detail distal end perspective view of the radially outward surfaces of the distal end region of the shaft shown in FIG. 2D;

FIG. 2G shows the device of FIGS. 2A-2F having a movable inner component extending through a lumen of the tube;

FIG. 3A is a detail side view of a distal end region of a shaft for use with the device of FIG. 2A showing the disruptor configuration;

FIG. 3B is a detail distal end perspective view of the radially outward surfaces of the distal end region of the shaft shown in FIG. 3A;

FIG. 3C is a detail distal end perspective view of the radially inward surfaces of the distal end region of the shaft shown in FIG. 3A;

FIG. 4A is a cross-sectional partial view of a distal end region of an implementation of a device for removing tissue from an eye;

FIG. 4B is a detail distal end perspective view of the distal end region of the shaft shown in FIG. 4A showing the disruptor configuration on a radially outward surface and radially inward surface;

FIG. 4B-1 is a detail view of the radially outward surface of the device of FIG. 4B;

FIG. 4C is a detail distal end perspective view of the distal end region of the shaft shown in FIG. 4A showing the disruptor configuration on a radially inward surface;

FIG. 5A is a side view of an implementation of a shaft having a tissue disruptor for use with any of the devices described herein;

FIG. 5B is a detailed view of the shaft of FIG. 5A taken along circle A;

FIG. 5C is a detailed view of the shaft of FIG. 5B taken along circle B;

FIG. 6A is a perspective view of an implementation of a shaft having a tissue disruptor for use with any of the devices described herein;

FIGS. 6B-6C are detailed view of the distal end region of the shaft of FIG. 6A;

FIG. 6D is a side view of the distal end region of the shaft of FIG. 6A;

FIG. 6E is a side view of a distal end region of a shaft having a tissue disruptor for use with any of the devices described herein;

FIG. 6F is a side view of a distal end region of a shaft having a tissue disruptor for use with any of the devices described herein;

FIGS. 6G-6I are side views of a distal end region of a shaft having a tissue disruptor for use with any of the devices described herein;

FIGS. 7A-7C are detail views of a distal end region of a device having a tissue disruptor;

FIG. 8A is a distal end region of a tissue disruptor for use with any of the devices described herein;

FIG. 8B is a detail view of the tissue disruptor in FIG. 8A taken at circle B;

FIG. 9A is a distal end region of a tissue disruptor for use with any of the devices described herein;

FIG. 9B is a detail view of the tissue disruptor of FIG. 9A taken at circle A;

FIG. 10A is a distal end region of an implementation of an introducer tube;

FIG. 10B is a distal end region of an implementation of an introducer tube;

FIG. 10C is a distal end region of a device showing the tissue disruptor shaft extending outside an introducer tube having a 90-degree bend;

FIG. 10D is a distal end region of a device showing the tissue disruptor shaft extending outside an introducer tube having a 120-degree bend;

FIG. 10E is a distal end region of device showing the tissue disruptor shaft extending outside an introducer tube having a 105-degree bend;

FIG. 10F is the introducer tube of FIG. 10B having a curve as shown in FIG. 10D;

FIG. 11 is a schematic illustrating a stiffening sleeve for the shaft of the device described herein;

FIG. 12A is a distal end region of a tissue disruptor for use with any of the devices described herein;

FIG. 12B is a detail view of the tissue disruptor of FIG. 12A taken at circle B;

FIG. 12C is a detail view of the tissue disruptor of FIG. 12A taken at circle C;

FIG. 13A is a distal end region of a tissue disruptor for use with any of the devices described herein;

FIG. 13B is a detail view of the tissue disruptor of FIG. 13A taken at circle B;

FIG. 13C is a detail view of the tissue disruptor of FIG. 13A taken at circle C

FIG. 14A is a distal end region of a tissue disruptor for use with any of the devices described herein;

FIG. 14B is a detail view of the tissue disruptor of FIG. 14A taken at circle A.

It should be appreciated that the drawings herein are for illustration only and are not meant to be to scale.

### DETAILED DESCRIPTION

The present disclosure relates generally to the field of ophthalmics, more particularly to increasing aqueous drainage of the eye. In one specific application, for example, the devices and methods may be used to remove trabecular meshwork (with or without part of Schlemm's canal) to treat glaucoma and other conditions. The devices described herein can disrupt one or more tissues in the eye to encourage outflow of aqueous. For example, the devices described herein can disrupt the inner wall of Schlemm's Canal (i.e., the trabeculorhexis) without cutting, for example, by bluntly engaging, tearing, and/or shearing or otherwise modifying the trabecular tissue, such as by a disinsertion of the trabecular meshwork from its attachment to the sclera and surrounding gonio anatomy, which will be described in more detail below. The devices described herein may simultaneously disrupt tissue of the inner canal wall (i.e., the trabecular meshwork) and modify the outer canal wall (i.e., the sclera). For example, a distal portion of the device inserted into the anterior chamber of the eye can have a first protrusion extending radially inwardly relative to the eye and a second protrusion disposed radially outwardly relative to the eye. Positioning the distal portion adjacent the trabecular meshwork and advancing it along a circumferential contour of Schlemm's Canal can disrupt the trabecular meshwork with the first protrusion as the device is advanced and, at the same time, disrupt the outer wall of Schlemm's Canal with the second protrusion. The first protrusion can remove a portion of an inner wall of Schlemm's Canal by bluntly tearing or disinserting the trabecular meshwork tissue, without cutting, thereby removing the obstacle into Schlemm's Canal and thus, the canal itself. The second protrusion can cut, slit, abrade, shave, debride, micro-perforate, and/or otherwise modify or disrupt the outer wall (i.e., the remaining portion of the canal after disruption of the inner wall during advancement) in a canal-independent manner. In still further implementations, the devices described herein can be used to disrupt the inner wall prior to modification of the outer wall. For example, no enclosed canal may be present in the anterior angle along at least a portion of the circumference of the eye prior to the modification of the outer wall because the inner wall formed by the trabecular meshwork was disrupted during advancement of the disruptor. Alternatively, the disruptor can disrupt the trabecular meshwork during advancement without disrupting the outer wall. In this implementation, the outer wall is disrupted only during retraction thereby providing an asynchronous disruption with less resistance and friction between the tissues and the device. The modification of the outer wall also need not be limited to the outer wall of what would otherwise be Schlemm's Canal as the devices can be used to modify the scleral wall from above the supraciliary segment at a posterior limit to below the clear-corneal margin/limbus at an anterior limit. A relatively wide band of the eye can be modified with the tools described here. The modification to the outer wall that can be performed after or prior to excisional or incisional removal, ablation, or disruption of the trabecular meshwork can vary (e.g., thinning, cutting, abrading, microporation, stenting, debriding, and other tissue modifications known in ophthalmology). The outer wall disruption can be performed during advancement through Schlemm's canal before the inner wall is disrupted. The device in this implementation may cannulate Schlemm's canal as it is advanced along a circumferential segment of the canal while a radially outward projecting feature of the device engages with the outer canal wall. In some implementations, use of the device spares the trabecular meshwork such that only outer wall modifications occur and the trabecular meshwork is left substantially unchanged except for the initial entry point into the canal from the anterior chamber. An outer wall modification, such as scraping, debriding, cutting of the outer wall with a feature on the shaft 6 that bears against the outer wall is performed to enhance outflow and lower IOP without any disruption of the trabecular meshwork. The outer wall modification can occur in a single pass within the Schlemm's Canal.

The modifications and methods using the devices described herein will be described in more detail below. In some implementations the tissue disruptor for the outer wall can incorporate micro-serrations for outer wall thinning and canaloplasty to improve canalicular/trans-scleral outflow as will be described in more detail below. In some implementations, the tissue disruptor can be positioned on an outer dimension of the tool and yet disrupts the inner wall without impacting the outer wall due to a wedging effect as will be described in more detail below. The outer wall modification can also include disruption using RF (radiofrequency) ablation or other electro or heat ablation process through the architecture of the outer-facing surface of a disruptor. The outer canal wall includes selectively and/or collectively any of the anatomic structures that are positioned radially outward from the canal including the endothelial layer of the Schlemm's canal as well as the adjacent scleral tissue.

The devices described herein preferably provide minimally-invasive disruption of Schlemm's canal by disrupting one or both of the trabecular meshwork tissue and the outer wall of Schlemm's canal in a manner that is deliverable and removable from a cannula.

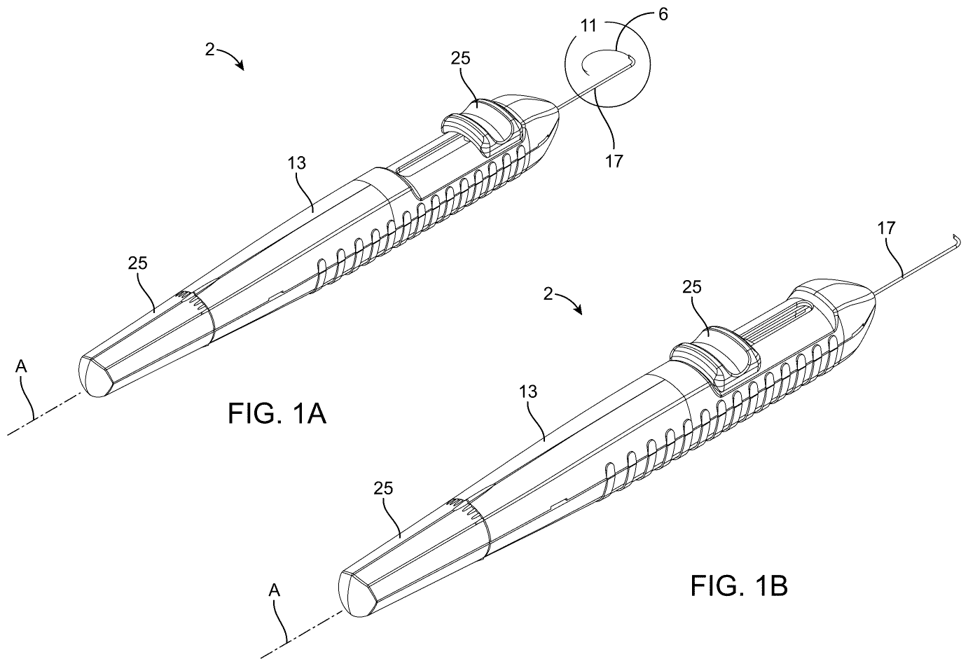

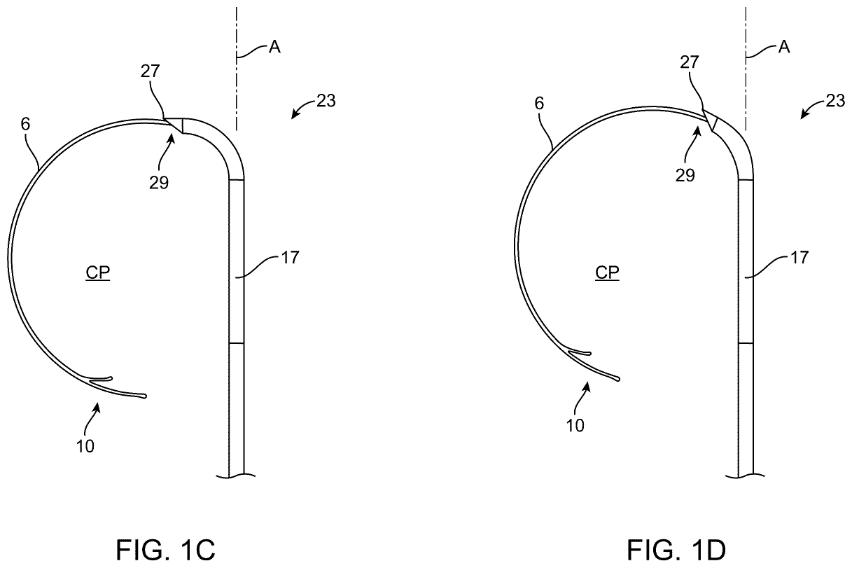

FIGS. 1A-1B illustrates an implementation of a device 2 for disrupting tissue within the anterior angle of the eye. The device 2 includes a proximal portion or housing 13 or hand piece coupled to a cannula or introducer tube 17 projecting from a distal end region of the housing 13. The device 2 also includes a tissue engager 10 on a shaft 6 that is configured to advance through the introducer tube 17 so that the tissue engager 10 extends outside the introducer tube 17 by degrees. The introducer tube 17 can be curved near a distal end region of the introducer tube 17 as shown in FIGS. 1A-1B. Optionally, the introducer tube 17 is substantially straight. The shaft 6 can curve along its distal end region upon extension through the introducer tube 17 as shown in FIG. 1A. Optionally, the shaft 6 is substantially straight and curves due to a flexibility of its material(s) and/or properties when it comes into contact with one or more eye tissues. The tissue engager 10 on the shaft 6 can have any of a variety of geometries as described herein, including tubular with a lumen extending through it (e.g., hypotube), flat ribbon without a lumen extending through it (e.g., Nitinol or stainless steel sheet cut to shape), round wire, and the like. The tissue engager 10 on the shaft 6 can be a surface feature on a radially inward surface of the shaft 6, on a radially outward surface of the shaft 6, or on both radially inward and radially outward surfaces of the shaft 6, and anywhere in between. The housing 13 can incorporate an actuator 25 to cause movement of the shaft 6, such as extension relative to the housing 13 to move the shaft 6 to extend further outside the introducer tube 17 and retraction relative to the housing 13 to move the shaft 6 further inside the introducer tube 17. The housing 13 can optionally incorporate an actuator 25 (which can be the same or a different actuator as the one to move the shaft 6) to move the introducer tube 17. The device 2 including the shaft 6 with the tissue engager 10, the introducer tube 17, and the housing 13 with one or more actuators 25 to move the shaft 6 will be described in further detail below.

The introducer 17 is preferably designed to reduce visual obstruction or interference within the surgical space. The outer diameter of the introducer 17 can be about 0.43 mm to about 0.47 mm, or no greater than about 0.60 mm, no greater than about 0.55 mm, or no greater than about 0.50 mm. The distal end region of the housing 13 is preferably tapered towards the location of the introducer tube 17. The tapered shape of the housing 13 in this region also reduces visual interference for a user while using the device 2 in the eye. A flexible shaft 6 is operatively coupled to the housing 13 and extends through the lumen 19 of the introducer tube 17. A distal end region of the flexible shaft 6 incorporates a tissue engager 10 used to disrupt tissue of the eye. The shaft 6 is configured to move relative to the housing 13 through the introducer tube 17 along a variety of lengths. The device 2 can incorporate any of a variety of shafts 6 as will be described herein, including a shaft 6 that is shape-set into a curve or that is substantially straight along its distal portion 11. Similarly, the device 2 can incorporate any of a variety of tissue engager 10 on the shaft 6, including any of the tissue engager 10 geometries described with respect to FIGS. 1A-1D, 2A-2G, 3A-3C, 4A-4C, 5A-5C, 6A-6I, 7A-7C, 8A-8B, 9A-9B, 11, 12A-12C, 13A-13C, and 14A-14B, each of which will be described in detail below. Similarly, the device 2 can incorporate any of a variety of introducer tubes 17, including any of the introducer tubes described with respect to FIGS. 1A-1D, 2A-2G, 3A-3C, 4A-4C, 5A-5C, 6A-6I, 7A-7C, 8A-8B, 9A-9B, 10A-10F, 11, 12A-12C, 13A-13C, and 14A-14B.

FIG. 1A illustrates the device 2 in a configuration in which the shaft 6 is fully extended from the distal end of the introducer tube 17. FIG. 1B illustrates the device 2 in a configuration in which the shaft 6 is fully retracted within the distal end of the introducer tube 17. When the shaft 6 is extended in use, the curved distal portion 11 of the shaft 6 changes the angle of the tissue engager 10 near the distal end region of the shaft 6 (and the orientation of the longitudinal axis of the shaft 6 at the distal end) relative to the housing 13. The angle can be changed by at least about 45 degrees relative to a longitudinal axis A of the housing 13 up to about 180 degrees relative to the longitudinal axis A of the housing 13. The distal portion 11 of the shaft 6 need not form a curve as illustrated in FIG. 1A and can extend from the introducer tube 17 remaining substantially straight and aligned with the longitudinal axis A.

The housing 13 includes one or more actuators 25 configured to move one or more portions of the device 2. One or more actuators 25 can be operatively coupled to the shaft 6 such that the shaft 6 can be translated forward and back relative to the housing 13 to extend and retract the shaft 6 from the introducer tube 17. FIGS. 1A-1B illustrate the housing 13 having an actuator 25 that is configured to control extension and retraction of the shaft 6 that is in the form of a slider on an upper surface of the housing 13.

The device 2 can additionally incorporate an actuator 25 in the form of a dial on the proximal end region of the housing 13. The dial on the proximal end of the housing 13 can turn at least 180 degrees up to 360 degrees around the longitudinal axis A of the housing 13 to control the direction of the distal opening 29 of the introducer tube 17 relative to the housing 13. The rotation of the dial can be unlimited such that the dial can continue to be rotated in the same direction as many times as a user desires. Rotation of the dial redirects the curve of the introducer tube 17 accordingly allowing a user to extend the shaft 6 from the introducer 17 in a clockwise or a counterclockwise direction (or any direction in between) relative to the hand of the user upon advancement of the actuator 25. The dial can incorporate one or more detents at circumferential locations around the axis A. The detents provide a user with tactile feedback of the degree of rotation of the introducer tube 17 around the axis A without the user needing to look at the housing 13 during adjustment. The housing 13 can also incorporate one or more indicators providing visual and/or tactile feedback of the rotation of the dial for improved user experience. Any of a variety of configurations of actuator 25 are considered herein. The one or more actuators 25 can include a button, slider, dial, or other actuator or combination of actuators.

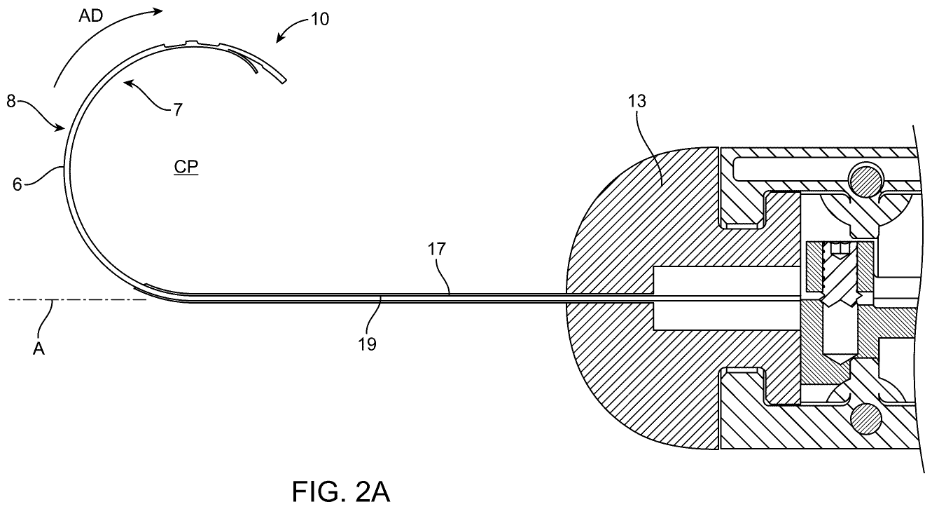

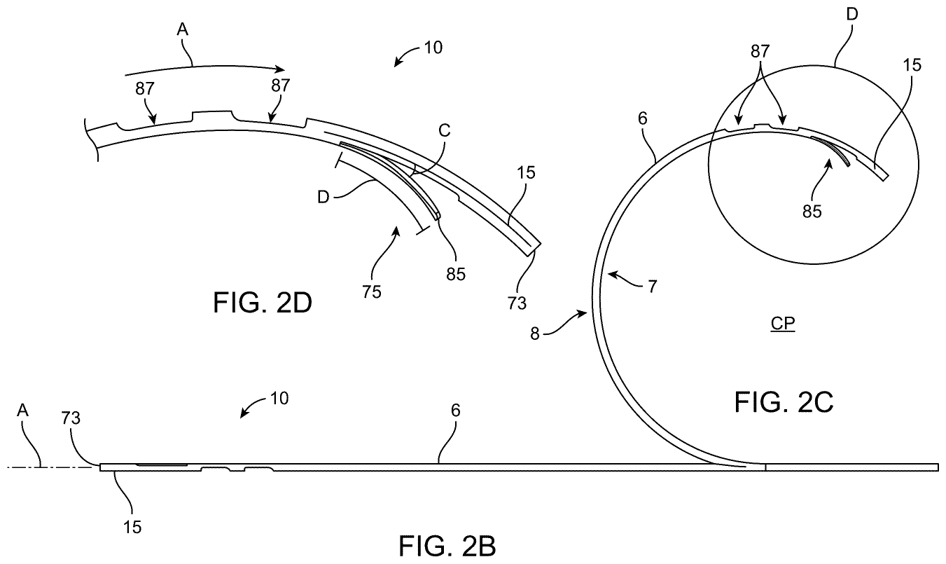

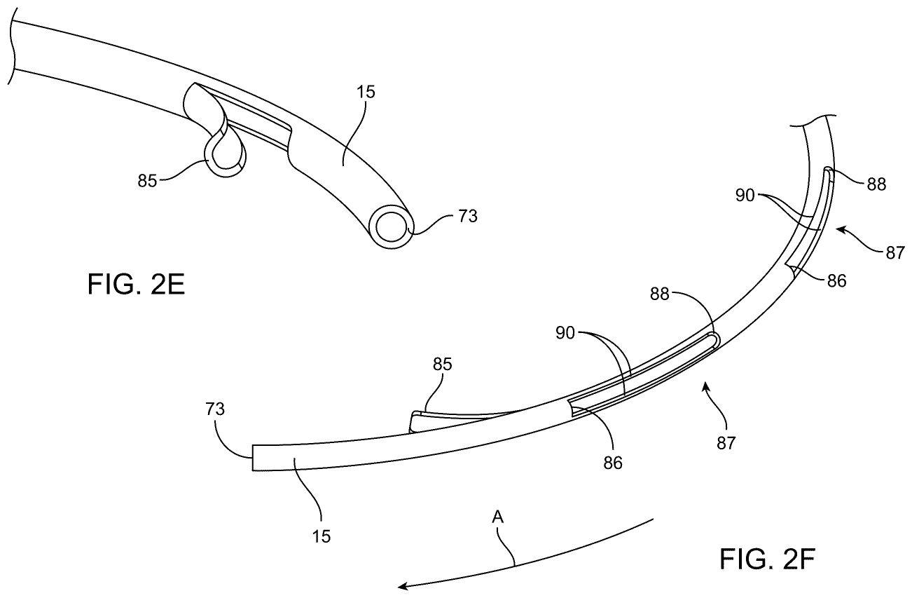

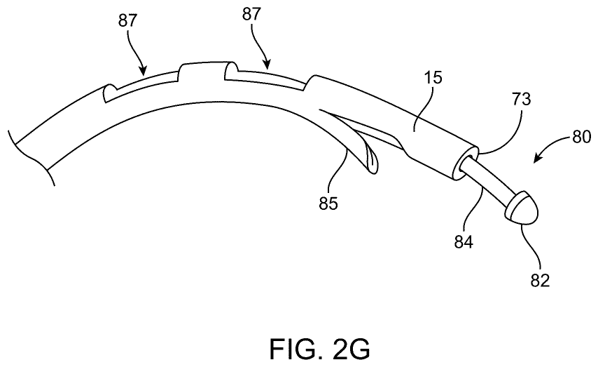

Again, with respect to FIGS. 1A-1B and FIGS. 1C-1D, the introducer tube 17 coupled to and extending distal from the housing 13 can be a tubular element having a lumen 19 extending through it such that the elongate shaft 6 extends through the lumen 19 of the introducer tube 17. The introducer tube 17 can be a stiffer component compared to the elongate shaft 6 extending through it such that the shaft 6 takes on a shape of the introducer tube 17 when retracted inside the introducer tube 17. The introducer tube 17 can be a substantially rigid tube. In an implementation, the introducer tube 17 can be a stainless-steel tube and the shaft 6 can be a flexible guidewire, tube, or cut sheet. The material of the shaft 6 can vary including Nitinol, stainless steel, plastic, such as polyimide film. The shaft 6 can take on a pre-set shape when extended relative to the introducer tube 17 (see FIG. 2C), but once retracted relative to the housing 13 to enter the lumen of the introducer tube 17, the shaft 6 can take on the shape of the introducer tube 17 (see FIG. 2B). For example, at least a portion of the introducer tube 17 can be relatively straight and extend along the single longitudinal axis A of the housing 13. The curved distal portion 11 of the shaft 6 can take on a straightened and biased condition upon withdrawal into the introducer tube 17. Alternatively, the curvature of the introducer tube 17 can be similar the curvature of the shaft 6 so that retraction of the shaft 6 into the introducer tube 17 does not substantially bias the shaft 6. As discussed elsewhere herein, the shaft 6 need not be shape-set to have a curve.

Again, with respect to FIGS. 1A-1B and 1C-1D, the introducer tube 17 can be sized so that the tissue engager 10 near the distal end region of the shaft 6, upon full retraction of the shaft 6 relative to the introducer tube 17, remains external to the lumen 19 of the introducer tube 17. The tissue engager 10 can bottom out against the distal end 27 of the introducer tube 17. In other implementations, the introducer tube 17 (and/or tissue engager 10) is sized so that the tissue engager 10 can be withdrawn fully inside the lumen 19 when the shaft 6 is fully retracted. In still further implementations, the introducer tube 17 can have a size smaller than a maximum outer diameter of the tissue engager 10, but is configured to accommodate the maximum outer diameter, such as by expanding or otherwise changing shape, upon full proximal retraction of the shaft 6 into the lumen 19. For example, the distal end region of the introducer tube 17 can incorporate one or more slits or slots through its wall so that as the tissue engager 10 is retracted and abuts against the distal edge 27 of the introducer tube 17, the walls of the introducer tube 17 flex outward or otherwise move to enlarge a lumen size of the introducer tube 17 to fully envelope the tissue engager 10. When the tissue engager 10 is fully enveloped within the lumen 19 of the introducer tube 17, the distal end 27 of the introducer tube 17 can be inserted through the trabecular meshwork to initiate the disruption. In still further implementations, the tissue engager 10 can be designed to collapse into a smaller dimension upon retraction into the lumen of the introducer tube 17 and to expand into a larger dimension when extended from the lumen of the introducer tube 17 (see, e.g., FIGS. 1C-1D, 2A-2G, 3A-3C, 4A-4C, 7A-7C, 8A-8B, 9A-9B, 12A-12C, 13A-13C, and 14A-14B).

The outer diameter of the distal end region of the introducer tube 17 that is designed to insert into the eye is preferably small enough to insert through a corneal incision without causing problems with the incision or requiring the incision to be too large. The outer diameter of the introducer tube 17 is generally as small as possible, but not so small that it interferes with movement of the shaft 6 extending through its lumen 19. Thus, the introducer tube 17 is sufficient in size to receive at least a region of the shaft 6. As mentioned above, some implementations of the shaft 6 can be fully withdrawn into the lumen 19 of the introducer tube 17 including the tissue engager 10. Thus, the inner diameter of the introducer tube 17 can be sufficiently large to receive the tissue engager 10 near the distal end region of the shaft 6. The smaller the outer diameter of the shaft 6, the smaller the outer diameter of the introducer tube 17 can be. In some implementations, the outer diameter of the introducer tube 17 is at least about 0.35 mm up to about 1.2 mm. In some implementations, the outer diameter of the introducer tube 17 is about 0.45 mm up to about 0.65 mm and the inner diameter of the introducer tube 17 is about 0.35 mm up to about 0.45 mm. The introducer tube 17 can be a stainless-steel tube, for example, a 0.022″ (0.56 mm OD) tube or between 0.020″ (0.51 mm OD) and 0.028″ (0.71 mm OD) or having a size configured to insert through a clear corneal incision less than about 2.75 mm, or less than about 2.5 mm, or about 1 mm.

The outer diameter of the introducer tube 17 is preferably about 0.43 mm-0.47 mm (inner diameter of about 0.33 mm-0.37 mm) to reduce visual obstruction within the surgical space. The outer diameter of the introducer tube 17 in this range is small enough to allow the introducer tube 17 to be pressed into and through the trabecular meshwork prior to advancing the shaft 6 relative to the tube 17. Positioning the distal end of the introducer tube 17 into Schlemm's Canal can improve stability during advancement of the shaft 6 through the Canal. The shaft 6 can be fully within the lumen of the introducer tube 17 upon insertion of the distal end of the introducer tube 17 within the canal so that the tip of the shaft 6 need not penetrate the trabecular meshwork on its own and can rely instead upon the introducer tube 17 to penetrate the trabecular meshwork. The larger inner diameter introducer tube 17 (e.g., about 0.41 mm) can have a greater wall thickness (e.g., about 0.08 mm) compared to the smaller inner diameter introducer tube 17 (e.g., about 0.36 mm) having a wall thickness of about 0.05 mm.

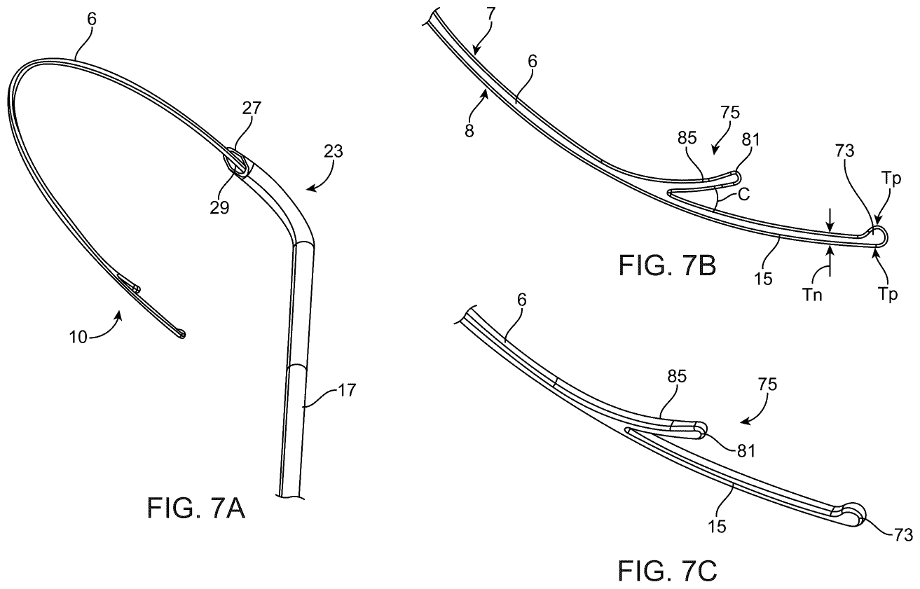

Still with respect to FIGS. 1A-1B and 1C-1D, the distal-most end of the introducer tube 17 can be designed to facilitate engagement into the canal, but is preferably not sharp. The distal edge 27 surrounding the distal opening 29 from the lumen 19 is preferably rounded without any sharp edges. The shape of the distal opening 29 can be circular. The shape of the distal opening 29 can be non-circular. For example, the distal opening 29 is formed at a distal end of the introducer tube 17 at an oblique angle such that the shape of the distal opening 29 is slightly elongate, elliptical, oval, or egg-shaped (see FIGS. 1C-1D, 7A). The bevel angle of the distal opening 29 of the introducer tube 17 can be about 20 degrees up to about 60 degrees.

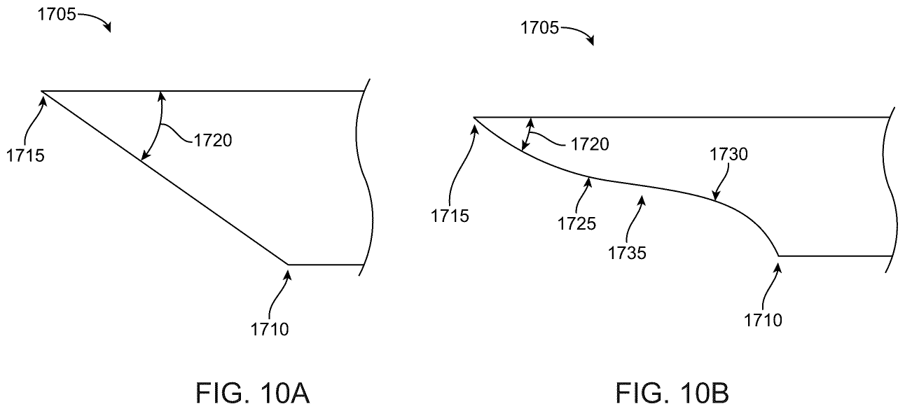

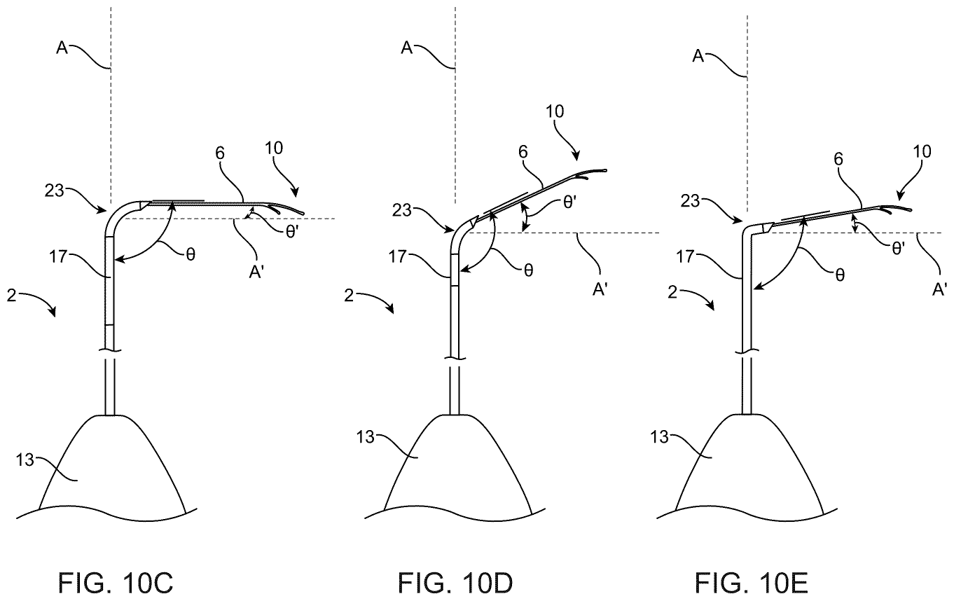

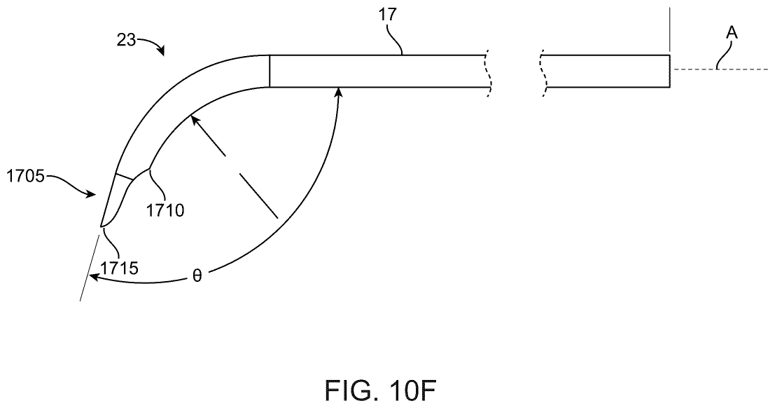

FIG. 10A shows a beveled tip 1705 of an introducer tube 17 forming the distal opening 29 from the lumen 19 having a heel 1710, a distal point 1715, and a length between the heel 1710 and distal point 1715. The length of the bevel tip 1705 from the heel 1710 to the distal point 1715 can be about 0.8 mm and form an angle 1720 that is about 35 degrees. FIG. 10B shows another tip 1705 of an introducer tube 17. The tip 1705 is shaped to form a piercing and introducing spatula. The length of the tip 1705 from the heel 1710 to the point 1715 can be slightly longer, such as about 1.00 mm. The angle 1720 of the beveled tip 1705 can be similar as the version shown in FIG. 10A (e.g., about 35 degrees) and have a thickness of about 0.08 mm. The bevel tip 1705 can have a first region 1725 forming a first curve and a second region 1730 forming a second curve. The first curve can have a different radius of curvature than the second curve. For example, the first curve can have a radius of curvature that is larger and the second curve can have a smaller radius of curvature that is smaller (e.g., about 0.80 compared to about 0.50). The first curve of the first region 1725 can extend from the distal point 1715 to a transition point 1735 between the first region 1725 and the second region 1730. The second curve of the second region 1730 can extend from the transition point 1735 to the heel 1710.

The introducer tube 17 of the devices described herein can be substantially straight and extend along the longitudinal axis A of the housing 13 from its proximal end to its distal end 27. Preferably, the introducer tube 17 incorporates a curve away from the longitudinal axis A along at least a portion of its length forming an angle relative to the longitudinal axis A. The curve can form an angle Θ that is between about 90 degrees up to about 170 degrees, the angle Θ being between the plane of the distal-most tip of the introducer tube 17 to a plane of the outer surface of the introducer tube 17 proximal to the curved distal end region 23. FIGS. 1A-1D and also FIGS. 10C-10F illustrate introducer tubes 17 with a curved distal end region 23. FIG. 1C (and also FIG. 10C) shows a curved distal end region 23 that forms an angle Θ that is about 90-degrees relative to the longitudinal axis A of the proximal region of the introducer tube 17 and has a radius of curvature that is about 1.8. FIGS. 1D (and also 10D) shows a curved distal end region 23 that forms an angle Θ) that is more obtuse than what is shown in FIG. 1C. Angles Θ that are too obtuse, such as about 120 degrees as shown in FIG. 1D or greater may cause the tissue disruptor shaft 6 to extend too deeply (steeply) into Schlemm's Canal. This can cause the disruptor on the radially inward side of the shaft 6 being inadvertently buried inside of the canal so that it is less capable or incapable of disrupting the trabecular meshwork. FIGS. 10C-10E illustrate how the angle Θ of the curved distal end region 23 of the introducer tube 17 impacts the angle of extension Θ′ relative to an axis A′ perpendicular to the longitudinal axis A of the shaft 6 upon extension from the distal end region 23 of the introducer tube 17. FIG. 10C shows a curved distal end region 23 that forms an angle Θ that is about 90-degrees relative to the longitudinal axis A of the proximal region of the introducer tube 17. FIG. 10D shows a curved distal end region 23 that forms an angle Θ that is about 120-degrees relative to the longitudinal axis A of the proximal region of the introducer tube 17. FIG. 10E shows a curved distal end region 23 that forms an angle Q that is about 105-degrees relative to the longitudinal axis A of the proximal region of the introducer tube 17. The tissue disruptor shaft 6 extending from the introducer tube 17 having an angle Θ that is about 90 degrees (FIG. 10C) up to about 105 degrees (FIG. 10E) extends at an angle of extension Q′ relative to an axis A′ perpendicular to the longitudinal axis A that is less than about 45 degrees, or about 10-40 degrees, preferably about 20-30 degrees and provides the proper depth of penetration for the tissue disruptor to engage with the trabecular meshwork upon insertion of the canal. In contrast, the tissue disruptor shaft 6 extending from the introducer tube 17 having a more obtuse angle Θ that is 120 degrees extends at an angle Θ′ relative to an axis A′ perpendicular to the longitudinal axis A that is about 45 degrees or greater (see FIG. 10D) and provides a depth of penetration of the tissue disruptor that is more prone to become buried within the canal. Thus, the devices described herein preferably incorporate a curved distal end region 23 of the introducer tubes 17 that forms an angle Θ relative to the longitudinal axis A of the proximal region of the introducer tube 17 that is between 90 and 120 degrees, preferably about 105-degrees.

The curved distal end region 23 of the introducer tube 17 facilitates insertion of the device 2 into the trabecular meshwork and to direct the tissue engager 10 of the shaft 6 in the desired direction along the anterior angle. The introducer tube 17 can have a curve that is at least 1.5 mm radial curvature up to about 10 mm radial curvature for tangential deployment of the shaft 6, preferably about 2.0 mm. The radial curvature of the introducer tube 17 preferably does not exceed 10 mm radius. The curved distal end region 23 has a larger radius of curvature of about 1.9. The angle Θ between the plane of the distal-most tip of the introducer tube 17 to the plane of the outer surface of the introducer tube proximal to the curved distal end region 23 can be at least about 90 degrees, at least about 95 degrees, at least about 100 degrees, at least about 105 degrees, at least about 110 degrees, and less than about 120 mm degrees. A more obtuse bend of about 120 degrees can result in the shaft 6 extending from the introducer tube 17 at an angle Θ that is too obtuse resulting in penetration into the canal that is too deep or too steep such that the disrupting feature of the shaft 6 gets inadvertently buried inside the canal and is unable to properly disrupt the trabecular meshwork.

In some implementations, the introducer tube 17 can be rotated relative to the housing 13 using an actuator 25 of the housing 13 such as the dial on the housing 13 as discussed above or by rotating the housing 13 itself. Rotation of the tube 17 can direct the tissue engager 10 to access a different band around the eye. For example, the introducer tube 17 can be rotated in a first direction relative to the housing 13 to direct the distal opening 29 from the lumen 19 anteriorly towards the limbus such that advancing the tissue engager 10 can perform a modification of this band of tissue. The introducer tube 17 can be rotated in a second direction relative to the housing 13 to direct the distal opening 29 from the lumen 19 posteriorly towards the ciliary body such that advancing the tissue engager 10 can perform a modification of this band of tissue. The device can also incorporate an actuator configured to move the introducer tube 17 and/or the shaft 6 along the longitudinal axis as well as around the longitudinal axis.

The actuator to rotate the introducer tube 17 can be a dial on a rear end of the housing 13 configured to rotate the introducer tube 17 in clockwise and counter-clockwise directions (see FIGS. 1A-1B). The dial can be formed by a rear section of the housing 13 that rotates around a longitudinal axis A of the housing 13 relative to a front section of the housing 13. The rear section can rotate, for example, up to about 180 degrees, and cause the introducer tube 17 to rotate a corresponding around the longitudinal axis. The introducer tube 17 can be rotated to create a larger disruption with the tissue engager 10. As an example, a surgeon can first deploy the shaft 6 and the tissue engager 10 from the introducer tube 17 counter-clockwise up to about 180 degrees. The shaft 6 can be withdrawn back inside the introducer tube 17 and the introducer tube 17 turned by 180 degrees relative to the housing 13 using the dial. The shaft 6 can then be advanced from the introducer tube 17 clockwise up to 180 degrees. The two advancements in counter-clockwise and clockwise directions can provide up to a 360-degree disruption, if desired. The dial configured to rotate the introducer tube 17 can provide a greater freedom for a user to orient the introducer tube 17 relative to the housing 13 depending on whether a right eye or a left eye is being treated and/or what approach is being used. The orientation of the introducer tube 17 to the housing 13 can be adjusted to suit a user's preferred access angle and location. The dial can allow for any of a variety of incremental degrees of rotation. The dial can provide a smooth feel during rotation or can provide tactile and/or auditory feedback as to the number of degrees the dial has been moved around the longitudinal axis. The introducer tube 17 can also be substantially fixed relative to the housing 13 such that it does not rotate or move relative to the housing 13.

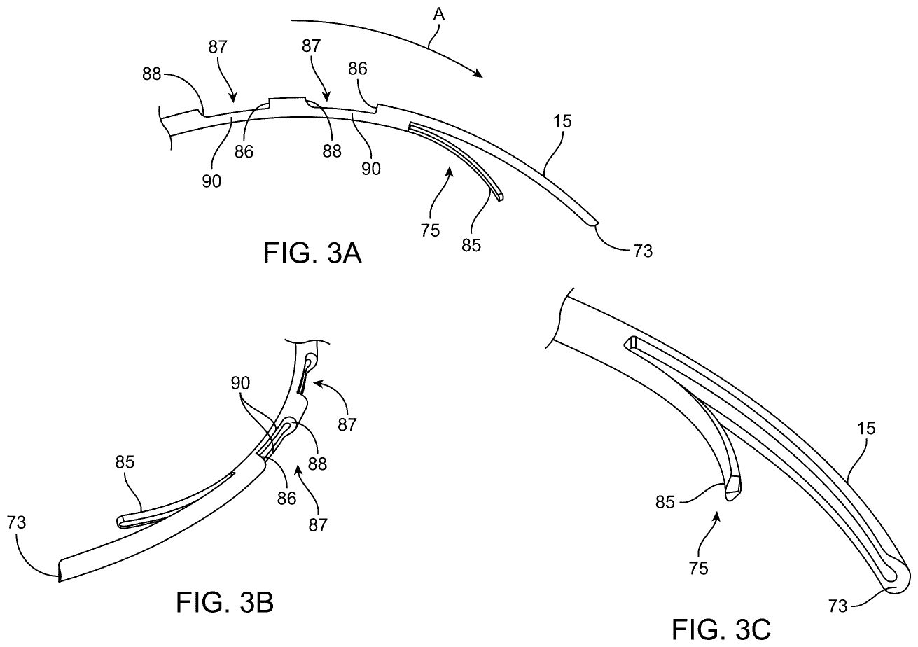

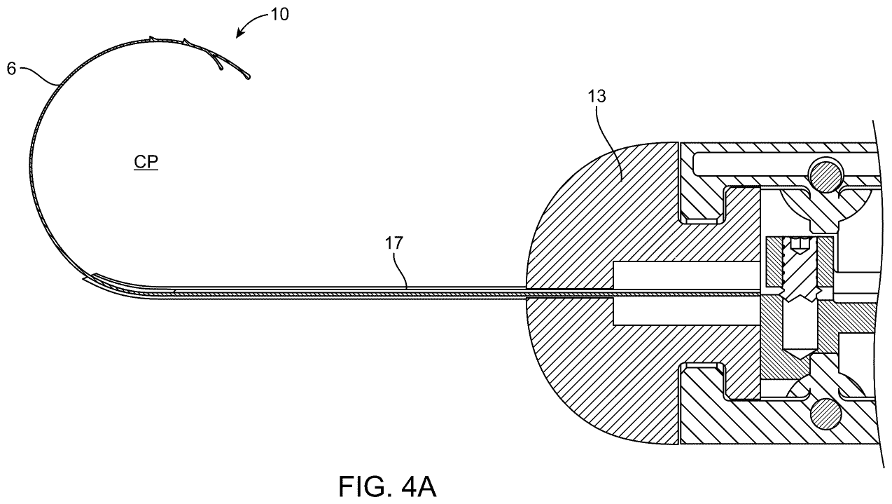

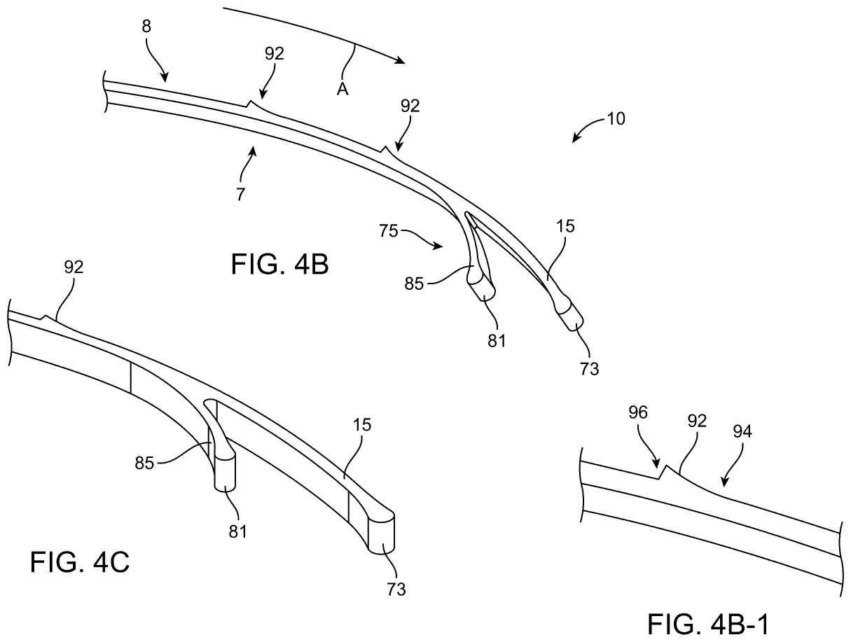

The shaft 6 can be a flexible material, such as Nitinol. The shaft 6 can also be formed of other materials, such as stainless steel, polyimide, or another plastic. The material can be in the form of a wire, such as a guidewire, a tube, sheet, or ribbon. FIGS. 2A-2F and FIGS. 3A-3C illustrate shafts that are in the form of a cut tube. FIGS. 4A-4C, 5A-5C, 6A-6I, 7A-7C, 8A-8B, 9A-9B, 12A-12C, 13A-13C, and 14A-14B illustrate shafts that are in the form of a flexible ribbon. The elongate shaft 6 may have a circular or non-circular cross-sectional shape, such as a square or rectangular cross-sectional shape. FIGS. 2A-2F and 3A-3C illustrate shafts 6 with circular cross-sectional shape whereas FIGS. 4A-4C, 5A-5C, 6A-6I, 7A-7C, 8A-8B, 9A-9B, 12A-12C, 13A-13C, and 14A-14B illustrate non-circular cross-sectional shaped shafts 6.

The shaft 6 can be formed of a super-elastic material that is shape-set. For example, the shaft 6 can have a pre-set curved shape forming a curved distal portion 11 having a radial curvature (see FIGS. 1A, 2A, 2C, 4A, 5A-5B, 6A, and 8A). The curved distal portion 11 can form a semi-circle having a contour or assume a radius of curvature that is like the limbus architecture of the eye. The radial curvature can approximate a circle having a diameter of 5-20 mm, 8-18 mm, 10-13 mm, or about 11-12 mm. The shaft 6 can have different curvatures and/or diameters along its length. The differential curvatures/diameters as the shaft 6 extends can allow optimal and segmental wall interface between the hardware and the eye wall. When the shaft 6 is extended in use, a curved distal portion 11 of the shaft 6 changes the angle of the tissue engager 10 near the distal end region of the shaft 6 (and the orientation of the longitudinal axis of the shaft 6 at the distal end) relative to the housing 13. The angle can be changed by at least about 45 degrees relative to a longitudinal axis A of the housing 13 up to about 180 degrees relative to the longitudinal axis A of the housing 13.

The shaft 6 may also have different flexibility along its length, such as by spiral cuts, scalloping, thinning of the shaft material along its length. The differences in curvature, diameter, and/or flexibility along the length of the shaft can aid in providing optimal traction and balance between forward momentum (e.g., during inner wall disruption), outward pressure (e.g., during outer wall modification), and flexibility.

In some implementations, the radial curvature of the distal portion 11 of the shaft 6 can be greater than the curvature of the limbus for sub-limbal gonio modification of the scleral wall. The slightly larger radial curvature ensures there is a bit of outward angular disposition of the shaft 6 such that it passively rides along the outer wall of the canal as the shaft 6 is extended and retracted. The diameter of the arc span of the flexible shaft 6 may have a memory shape that is at least about 10 mm, at least about 11 mm, at least about 12 mm, or at least about 13 mm in diameter. In some implementations, the diameter of the arc span of the flexible shaft 6 may have a memory shape slightly exceeding the diameter of the average eye limbus or about 13 mm in diameter. The slightly larger diameter can allow for the shaft 6 to impart a slight radially outward force on the eye tissue as the shaft 6 is extended relative to the housing 13 and travels along the anterior angle. The shaft 6 can abut against the firm, outside scleral wall so that the outer wall further guides the device 2 as the tissue engager 10 near the distal end of the shaft 6 is advanced distally. The curved distal portion 11 of the shaft 6 can extend for an angle of greater than 135 degrees, greater than 160, greater than 180 degrees, greater than 200 degrees, greater than 240 degrees or more. The curved distal portion 11 can extend for an angle of between 160 and 225 degrees. In still further implementations, the curved distal portion 11 can extend a full 360 degrees.

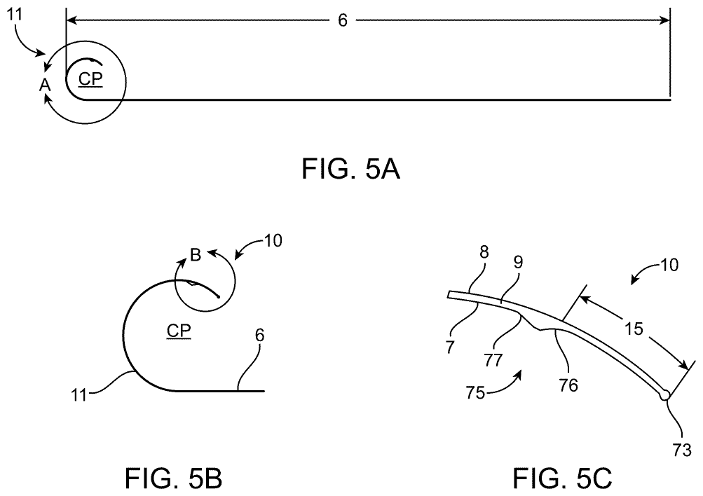

A central plane CP (see FIGS. 5A and 6A and 8A) can be defined as the plane containing the circular shape of Schlemm's Canal. The shaft 6 can be a flexible memory shaped material configured to substantially conform to the contour of the eye. The distal portion 11 can be shape-set to define the plane of curvature or can be flexible so as to take on the plane of curvature while in use. The distal portion 11 of the shaft 6 can have a curved shape that lies in the plane of curvature in use that is aligned with the plane on which the circular Schlemm's Canal lies. It should be appreciated that where a central plane CP is referred to herein that the central plane CP can be regarding a shape-set curvature of the distal portion 11 of the shaft 6 or a curvature of the distal portion 11 of the shaft 6 that occurs during use of the shaft 6 in the eye. For example, the shafts 6 described herein can be formed of a material or a combination of materials that are sufficiently flexible at least at a distal end region of the shaft 6 that allows the shaft 6 to conform to the curvature of Schlemm's canal as the shaft 6 extends out the introducer tube. A central plane CP of the distal portion 11 of the shaft 6 is created as the shaft 6 extends further into the eye and conforms to the curve of the eye.

The elongate shaft 6 may be flexible and resilient to provide a “soft” feel during use with the shaft 6 being elastically deflected and deformed in use. Specifically, the shaft 6 may be resilient relative to forces exerted against the tissue engager 10 in the advancing direction AD. The shaft 6 is not so flexible that it is not pushable along eye tissue. The shaft 6 can have a light spring-load in the advancing direction AD as it is advanced. The distal portion 11 of the shaft 6 can also provide a resilient response in a direction perpendicular to the advancing direction AD and lying in the plane of curvature CP. The shaft 6 may develop a spring load in the advancing direction AD and in a radially outward direction relative to the visual axis of the eye. In this manner, the radially outward force can cause the tissue engager 10 coupled to a distal end region of the shaft 6 to slide against the sclera (i.e., outer wall of Schlemm's Canal) to stabilize the tissue engager 10. Stated another way, as the tissue engager 10 is moved through the trabecular tissue, the shaft 6 can apply a radially outward force on the tissue relative to the axis of the eye. In some implementations, the radially outward force on the tissue is provided by the distal portion 11 of the shaft 6 being shape-set into a curve. As discussed herein, the distal portion 11 of the shaft 6 need not be shape-set into a curve and instead conform to the curve of the anatomy it is advanced into. Where the shaft 6 is not shape-set into a curve, but instead takes on a curve during use, the properties of the shaft 6 (e.g., thickness, width, and/or material(s), etc.) influence the force applied to the outer wall (or not applied to the outer wall) as well as the curvature the shaft conforms to (or does not conform to). While the shaft 6 is pushable and can apply a radially outward force, the resilient nature of the shaft 6 can limit or prevent excessive forces or displacement from being applied to the eye inadvertently. For example, the shaft 6 may be made of a metal and may be a superelastic material, such as Nitinol or another non-Nitinol metal, which provides a wide range of elastic response. The shaft 6 can also be plastic (extrusion or molded) or another non-metal material.

The shaft 6 can have an outer dimension of 100-1100 microns. In some implementations, the shaft 6 is about 0.250 mm×0.100 mm. The shaft may be 0.15 mm diameter wire and may be 0.10 to 0.25 mm. The wire can be a Nitinol wire. The shaft 6 can incorporate one or more cuts along its length to provide flexibility. For example, the shaft 6 can be a spiral-cut tube extending along one or more regions between the proximal and distal ends of the shaft 6. The spiral-cut tube, such as a Nitinol tube, can have a slightly larger outer diameter than the wire, for example about 0.175 mm. The shaft can be a flat sheet of material, such as Nitinol, cut or machined to size to achieve a particular dimension to the shaft and for the geometry of the tissue engager at the distal end region (which will be described in detail below). For example, the flat sheet of material may be between 0.10-0.15 mm thick and about 0.10-0.15 mm wide, or about 0.100 mm thick and 0.250 mm wide. The shaft 6 can be a laser-cut tube (e.g., plastic or metal including stainless steel or Nitinol), for example, a 0.250 mm outer diameter tube. The shaft 6 can have a maximum outer diameter sized to fit within an inner diameter of the introducer 17. The introducer 17 can have an outer diameter of about 0.60 mm and an inner diameter of about 0.45 mm. The shaft 6 can have a maximum outer diameter sized to fit within the 0.45 mm inner diameter of the introducer 17. The shaft 6 can also have a maximum outer diameter or dimension that is larger than the introducer 17, such as at the tissue engager 10. In some implementations, the larger dimension of the tissue engager 10 remains external to the smaller dimension introducer 17. In other implementations, the larger dimension of the tissue engager 10 can be withdrawn into the smaller dimension introducer 17 by virtue of its collapsible configuration, which will be discussed elsewhere herein.

In some implementations, the shaft 6 is formed from a flat sheet of material that is shaped by cutting and/or micro-machining into an elongate element forming the tissue engager 10 of the shaft 6 (www.memry.com/laser-cutting). The sheet may be cut by a laser to the desired geometry and/or shape. The starting material may be sheet of material, such as Nitinol or stainless steel or polyimide, that is between about 75 microns and 550 microns thick or more preferably between about 100 microns and about 150 microns thick. A shaft 6 cut, shaped, or printed from the flat sheet need not have a round cross section although the sheet can be curled into a round cross-section, if desired. The sheet of material can be cut into an elongate shape having an inner and outer surfaces. The manufacturing process of the micro-interventional instrumentation components for use in the canal or within the anterior angle of the eye can include laser-printing and/or laser-shaping a flat sheet of super-elastic memory-shape material to dimensions that are as small as 5 microns up to about 5,000 microns. The manufacturing process needs no micromachining and/or molding and/or assembly of the components. The manufacturing process provides cost-effective automated or semi-automated manufacturing of gonio-disruptors, gonio-shafts, and/or gonio-probes in an assembly-free manner.

Where the shaft 6 is referred to herein as a “wire” it should be appreciated the shaft 6 may also be a tube or ribbon. As mentioned above, the shaft can have a non-circular cross-sectional shape. The non-circular cross-sectional shape can have a minor axis and a major axis, the major axis being within 30 degrees, and may be within 15 degrees, of perpendicular to the central plane. The major axis may be at least 20% larger than the minor axis. The minor axis may be less than 250 microns while the major axis may be larger than 250 microns. The shaft 6 may have an effective radius of 40 to 400 microns, or 50-300 microns, although different sizes and shapes may be used. The effective radius is the equivalent radius for a circle having the same cross-sectional area for a non-circular cross-section (such as elliptical, square, or rectangular).

The shaft 6 can have a variable stiffness by changing a length of the shaft 6 extending outside the lumen 19 of the introducer tube 17. The variable stiffness of the shaft 6 can be changed by at least at factor of 10 when moving between a first working position (e.g., retracted) and a second working position (e.g., extended) so that the first position with the smallest stiffness is at least 10 times smaller than the second position with the larger stiffness with both positions being operable to displace the tissue. The variable stiffness may be provided by retracting and extending the shaft 6 to change a length of the shaft 6 extending from the housing 13 outside the introducer tube 17. The first and second working positions may change the orientation of the distal end 73 of the shaft 6 by at least 45 degrees relative to the housing 13. The first and second working positions need not change the orientation of the distal end 73 of the shaft 6, such as with a straight shaft 6 that does not curve along its distal portion 11. The shaft 6 cross-section may be constant or may increase proximally to maintain a more consistent stiffness. For example, the stiffness may vary less than 30% for a curved portion that is extended and retracted to change the angle of the shaft 6 by at least 45 degrees.

As mentioned, the distal end region of the shaft 6 incorporates a tissue engager 10 that can include one or more disruptors to disrupt tissue of the eye. The disruptor of the tissue engager 10 can be formed as a non-cutting, blunt element projecting away from the longitudinal axis A or the center of the shaft 6 that is configured to engage tissue in the anterior angle, such as the trabecular meshwork and/or the outer wall of Schlemm's Canal, as the shaft 6 is advanced out from the introducer tube 17 along the anterior chamber angle. The tissue engager 10 can slide along an inner wall (or along an outer wall) of the Schlemm's canal. Tissue within the eye, such as the trabecular meshwork and/or outer wall of Schlemm's canal can be disrupted upon advancement, upon retraction, or upon a combination of advancement and retraction. The disruptor of the tissue engager 10 may be a blunt feature sized to span the trabecular meshwork to form a continuous non-cutting trabeculorhexis so that the tissue engager 10 stretches and tears the trabecular meshwork fibers as it follows the contour of the eye and may disinsert some of the tissue at the origin. The tissue engager 10 can be introduced into an anterior chamber of an eye positioned adjacent tissue in the anterior chamber angle, e.g., an inner or outer wall of Schlemm's Canal. The tissue engager 10 can be moved by advancing the shaft 6 in an advancing direction AD and parts of the trabecular meshwork removed by the disruptor, such as by bluntly tearing, stripping, and/or disinserting the trabecular meshwork tissue.

The configuration of the tissue engager 10 can vary depending upon the configuration of the shaft 6 (e.g., wire, tube, ribbon). In some implementations, the tissue engager 10 includes one or more disruptors 75 on an inward surface 7 of the distal end region of the shaft 6, sometimes referred to herein as a radially inward surface. In other implementations, the tissue engager 10 includes one or more disruptors 75 on an outward surface 8 of the distal end region of the shaft 6, sometimes referred to herein as a radially outward surface. In still further implementations, the tissue engager 10 includes one or more disruptors 75 on both the radially inward surface and radially outward surface 7, 8 of the distal end region of the shaft 6.

Radially inward refers to a surface that is facing towards an interior of the eye during use of the shaft 6 and radially outward refers to a surface that is facing away from an interior of the eye during use of the shaft 6. The radially inward surface may be a surface on a distal end region of the shaft 6 that is substantially curved in its resting state and, during use, faces towards an interior of the eye. The radially outward surface may be a surface on a distal end region of the shaft 6 that is substantially curved in its resting state and, during use, faces away from the interior of the eye. The radially inward surface may be a surface on a distal end region of the shaft 6 that is substantially straight in its resting state and, during use, faces towards an interior of the eye. The radially outward surface may be a surface on a distal end region of the shaft 6 that is substantially straight in its resting state and, during use, faces away from the interior of the eye.

Where the tools are described herein as having a tissue disruptor directed radially inward from the shaft or on a radially inward surface of the shaft, the tool can additionally incorporate a tissue disruptor or cutter projecting radially outward or on a radially outward surface of the shaft so that the inner wall modification to the trabecular meshwork can be performed simultaneously with the outer wall modification to the scleral tissue. The tools described herein can alternatively incorporate a tissue disruptor or cutter projecting radially outward from the shaft or on a radially outward surface of the shaft so that the outer wall modification can be performed separately from the inner wall modification, such as only during retraction of the shaft. For example, the shaft can incorporate a tissue disruptor on the distal end region that is on a radially inward surface and a radially outward surface of the shaft where the inward surface is the surface of the shaft facing toward a central axis of CP and the radially outward surface is the surface of the shaft facing outward from the central axis of CP. The tissue disruptor is designed to disrupt only the trabecular meshwork with the radially inward surface during advancement of the shaft relative to Schlemm's canal without disrupting the outer wall of Schlemm's canal and to disrupt only the outer wall of Schlemm's during retraction of the shaft with the radially outward surface. Thus, the inner wall modification can be performed as a first step (i.e., advancing the shaft) and the outer wall modification can be performed as a second step (i.e., retracting the shaft). While a single tool is described herein to perform the inner and outer wall modifications, it should be appreciated that the inner wall modification can be performed with a first tool to disrupt the trabecular meshwork and expose the outer wall of Schlemm's canal as a first step so that a second tool can be used to disrupt the outer wall as a second step. The tools described herein may also be designed to disrupt the outer wall of Schlemm's Canal to control intraocular pressure and spare or preserve the trabecular meshwork. For example, the shaft 6 need not be fabricated to include a disrupting feature on the radially inward region of the shaft 6 that would disrupt the trabecular meshwork. In this example, the shaft 6 would incorporate feature(s) only on the radially outward side of the shaft 6 that are arranged and designed to bear against the outer wall of Schlemm's canal. In order to access the outer wall of Schlemm's canal, however, such a system would penetrate the trabecular meshwork to enter the canal creating a limited disruption of the trabecular meshwork at the canal entry location. The shaft 6, however, need not disrupt the trabecular meshwork at all. The shaft can include any of a variety of features on the radially outward portion of the shaft to engage the outer wall of Schlemm's canal, such as to cut or score or otherwise modify the outer wall, during advancement of the shaft 6 through the Canal, retraction of the shaft 6 through the Canal, and/or both advancement and retraction and not tear or disrupt in any way the trabecular meshwork except for the location where Schlemm's Canal was entered.

Where a feature projects from or is located on a radially inward side of the shaft 6, the feature can be blunt or sharpened to disrupt the trabecular meshwork, which is a relative thin and delicate tissue type that is relatively easily disinserted. Where a feature projects from or is located on a radially outward side of the shaft 6, the feature can be sharpened, serrated, and/or abrasive to cut, debride, reduce, thin, and/or shave the tougher scleral tissue forming the outer wall of Schlemm's canal. Where a single tissue disruptor or cutter is described, more than a single tissue disruptor or cutter can be incorporated so that the plurality of tissue disruptors/cutters can create a micro-serration to the shaft 6 on either a radially inward surface of the shaft 6, a radially outward surface of the shaft 6, or both radially inward and radially outward surfaces of the shaft 6.

FIGS. 2A-2G illustrate an implementation of a device 2 having a tissue disruptor on a distal end region of a shaft 6 that is in the form of a tube. FIG. 2A is a cross-sectional partial view of the device 2 showing the shaft 6 extending out from the distal end of the introducer tube 17 and forming a curved distal portion 11. FIG. 2B is a side view of a shaft 6 for use with the device of FIG. 2A illustrating a straight shape setting of a cut tube. FIG. 2C is a side view of the shaft 6 illustrating a curved shape-setting of the cut tube forming the curved distal portion 11 with the radially outward side of the shaft 6 facing away from the center of the central plane CP and the radially inward side of the shaft 6 facing toward the center of the central plane CP. FIG. 2D is a detail side view of the distal end region of the shaft 6 of FIG. 2C showing the tissue engager 10.

The edges at the distal end 73 of the tubular shaft 6 can be rounded or smoothed forming an atraumatic tip that is configured for circumferential gonio-traction and allowing for the shaft 6 to slide better relative to the tissue. Alternatively, the device 2 can incorporate a movable inner component 80 sized to extend through the lumen of the tube 6 so an atraumatic tip 82 of the inner component 80 projects distally from the distal end 73 of the shaft 6. The tip 82 can be shaped to smooth the square edges of the distal end 73 of the shaft 6 (see FIG. 2G). The tip 82 can have a tapered or bullet-shaped feature that changes in outer diameter from a smaller outer diameter at a distal-most end and a larger outer diameter at a proximal end. The proximal end outer diameter can be substantially the same as the outer diameter of the distal-most end 73 of the shaft 6 so that the tip 82 provides a smooth transition to the outer diameter of the shaft 6 along a distal-facing surface of the shaft 6. The inner component 80 can be moveable relative to the shaft 6 so that the tip 82 can be extended distally away from the distal-most end 73 of the shaft 6 as shown in FIG. 2G and withdrawn proximally so the proximal end of the tip 82 abuts against the distal end 73 of the shaft 6. The tip 82 can be coupled to a smaller diameter control wire 84. The smaller outer diameter of the control wire 84 can allow for ease of movement between the shaft 6 and the wire 84 during use with less friction. In some implementations, the outer diameter of the proximal end of the tip 82 is sized to be fully withdrawn within the inner diameter of the shaft 6 and/or removed from the shaft 6. In this implementation, the outer diameter of the proximal end of the tip 82 is smaller than, but substantially similar to the inner diameter of the shaft 6. The difference between the outer diameter of the proximal end of the tip 82 and the inner diameter of the shaft 6 can vary, but is generally small enough to provide a smooth transition from the shape of the tip 82 to the shape of the shaft 6. The removable inner component 80 can provide additional rigidity to the shaft 6 when extending through the lumen of the shaft 6 and reduce rigidity of the shaft 6 upon removal from the lumen of the shaft 6. In some implementations, the proximal end of the tip 82 can be used to aid in catching debris between it and the distal end 73 of the shaft 6. For example, the tip 82 can be extended distally away from the distal end 73 of the shaft 6 and then withdrawn proximally capturing debris between it and the shaft 6 such as by pinching the debris at the interface and/or pulling the debris into the lumen of the shaft 6. The inner lumen of the shaft 6 can also be used to deliver one or more materials (e.g., viscoelastic, therapeutic agent, saline) out the distal opening 29 and/or deliver vacuum to aid in removing debris and material from the treatment site.

Again, regarding FIG. 2D and also FIGS. 2E-2F, the distal end 73 of the shaft 6 can be formed by a short segment of the tube extending distal to the disruptor 75 forming a distal guide member 15. The length of the distal guide member 15 can vary, such as from about 1 mm up to about 5 mm and can act as a probe tip for the tissue disruptor 75.

The distal end region of the shaft 6 can have a radially inward surface 7 and a radially outward surface 8. The distal end region of the shaft 6 can be curved as shown in FIGS. 2A-2G, or can be straight as described elsewhere herein. The tissue engager 10 can incorporate a tissue disruptor 75 formed on the radially inward surface 7 of the distal portion 11 of the shaft 6 in the shape of a seam ripper 85. The seam ripper 85 can be formed by laser-cutting the tube 6 along at least 3 edges to create a segment that can flex inward toward the longitudinal axis A and can flex outward away from the longitudinal axis A of or the center of the shaft 6. The seam ripper 85 in any of the implementations described herein can take on a collapsed configuration when flexed inward during delivery. The collapsed configuration of the seam ripper 85 can allow for the distal end region of the shaft 6 to be contained by the lumen of the introducer 17. The seam ripper 85 can also flex outward into an expanded configuration once disruption is desired and the seam ripper 85 is removed from constraint, such as extended outside the introducer tube 17. The length of the segment can vary as can the angle that the segment flexes away from the shaft 6. The seam ripper 85 on the radially inward surface 7 can be a blunt tissue-engaging feature without any cutting element that provides trabeculorhexis on advancement of the shaft 6 in a direction of arrow A (see FIG. 2D).

The distal end region of the shaft 6 can additionally incorporate a disruptor 75 on the radially outward surface 8 that can provide debridement of the outer wall of Schlemm's canal on retraction in the direction opposite of arrow A. The disruptor 75 on the radially outward surface 8 can be a debrider or other type of cutting element formed by one, two, or more discontinuities or windows 87 in the tubular shaft 6. Disruptors like debriders can effectively remove tissue of the outer canal wall thereby thinning it and enabling more drainage. The disruptor 75 on the radially outward surface can be “on plane” with the iris so that there is no tilt. Alternatively, the disruptor 75 can be positioned at an angle relative to the plane of the iris so that the disruptor 75 is tilted. The tube of the shaft 6 can have a lumen extending along a longitudinal axis and defined by a cylindrical wall. A first tissue disruptor on the radially inward surface 7 can be a segment of the cylindrical wall of the tube projecting inward at an angle relative to the longitudinal axis. The second tissue disruptor on the radially outward surface 8 can be a discontinuity forming a window in the cylindrical wall of the tube. FIG. 2D is a side view of the distal end region of the shaft 6 taken at circle D of FIG. 2C showing two windows 87 on the radially outward surface 8 just proximal to a location of the seam ripper 85 on the radially inward surface 7. FIG. 2F is a detail distal end perspective view of the radially outward surface 8 of the distal end region of the shaft 6 illustrating the configuration of the windows 87. The windows 87 can be formed by laser-cutting the radially outward surface 8 of the distal end region of the tubular shaft 6. The window 87 can have at least a leading edge 86 facing proximally and a trailing edge 88 facing distally. The window 87 can be generally rectangular or elongate so that the leading and trailing edges are joined by two lateral edges 90. The trailing edge 88 of the windows 87 that faces distally can be blunt to avoid cutting tissue upon advancement in the direction of arrow A (i.e., distal direction). The leading edge 86 that faces proximally can be sharp to cut tissue upon retraction in the direction opposite of arrow A (i.e., proximal direction). Thus, the windows form a tissue disruptor that is configured to disrupt tissue upon retraction of the shaft and not as the shaft is advanced along the circumferential contour of Schlemm's Canal as the seam ripper or other tissue disruptor on the radially inward surface 7 of the distal portion 11 disrupts.

The leading edge 86 of each window 87 need not be sharpened to debride the outer wall of Schlemm's. For example, the disruptor on the radially outward surface 8 of the shaft 6 can incorporate tines (not shown) that are coupled to the leading edge 86 of each window 87 and configured to project outward away from the long axis A of the shaft 6. The tines can be designed to flex inward toward the window 87 during advancement of the shaft (in direction of arrow A) as the shaft 6 is urged against the outer wall of Schlemm's and to flex outward away from the window 87 during retraction of the shaft 6 (opposite direction of arrow A) to catch on tissue thereby forming a one-way tissue disruptor.

The shaft 6 can be laser-cut to form the windows 87 and the seam ripper 85 and any other feature of the tissue disruptors. The laser-cut tube 6 can be shape-set prior to or after cutting to achieve the curve of the distal portion 11. The seam ripper 85 has an angle C and a length D (see FIGS. 2D or 7B). The length D of the seam ripper 85 can be about 1.5 mm-3.0 mm, preferably about 1.75 mm. The seam ripper 85 can be about 0.150 mm-2.00 mm wide, preferably about 0.196 mm wide. Each window 87 can be about 0.250 mm-0.400 mm wide, preferably about 0.393 mm wide. Each window 87 can be about 0.5 mm-1.5 mm long, preferably about 1 mm long. The windows 87 can be spaced from one another by about 0.25 mm-1.00 mm, preferably about 0.50 mm.

The laser-cut tube can additionally be electropolished to remove an outer layer of the metal and any microscopic imperfections in the finish that could impact the ability of the shaft 6, including the cut portions of the shaft 6 past and/or through the tissues. In some implementations, the shaft 6 is electropolished to reduce the overall thickness of the component by approximately 20 microns, 25 microns, 30 microns, or 35 microns up to about half the thickness of the shaft 6 along at least a portion of its length. Electropolishing of the shaft 6 formed from a flat piece of material and having a square or rectangular cross-section can cause the corners of the material to be rounded. Thus, a square cross-section shaft 6 may become more circular in cross-section and a rectangular cross-section shaft 6 may become more oval in cross-section upon electropolishing. The zone of electropolishing can extend along the shaft 6 from a location just proximal of the location of the disruptors to the distal-most end of the shaft 6. The zone of electropolishing can extend along the shaft 6 proximally along at least the portion of the shaft that is intended to enter the eye and/or come into direct contact with tissue.

The radially inward disruptor 75 (which can include the seam ripper 85 described above) preferably projects away from the long axis A or center of the shaft 6 to drag along the trabecular meshwork as the shaft 6 is advanced along the angle of the eye. The radially outward disruptor 75 can, but need not, project away from the long axis A or center of the shaft 6 as well. In the implementation shown in FIGS. 2A-2G and also FIGS. 3A-3C, the radially outward disruptor 75 include discontinuity in the shaft surface, such as the window 87, forming edges, such as the leading ledge 86, that can disrupt upon being dragged along the outer wall surface thereby debriding it.

FIGS. 3A-3C illustrates another implementation of a tissue disruptor 10 on a shaft 6 that is a tube. The distal end 73 of the shaft 6 can be formed by a short segment of the tube extending distal to the disruptor 75 forming a distal guide member 15 that is not fully tubular and is merely a segment of the tube. The length of the distal guide member 15 can vary as discussed above but is generally at least about 1 mm up to about 5 mm, preferably about 1.5 mm-1.75 mm, to act as a probe tip for the tissue disruptor 75. The segment can be hemicylindrical including at least about 25% up to about 75% of the full circumference of the tubular shaft 6 forming an arcuate portion (see FIG. 3C). The distal end of the hemi-cylindrical probe tip 73 can be angled to a sharpened tip to aid in insertion of the probe tip through the trabecular meshwork and into the Schlemm's canal.

The distal end region of the shaft 6 can have a radially inward surface 7 and a radially outward surface 8. The hemi-cylindrical region forming the probe tip 15 can transition moving proximally along the shaft 6 into the seam ripper 85 on the radially inward surface 7 of the distal portion 11 of the shaft 6. Thus, the seam ripper 85 and the hemi-cylindrical probe tip 15 can be formed by laser-cutting the tubular shaft 6 along at least 2 edges to create a segment that can flex outward away from the longitudinal axis A of the shaft 6. The length of the segment can vary as can the angle that the segment flexes away from the shaft 6. As with other implementations, the seam ripper 85 on the radially inward surface 7 can be a blunt tissue-engaging feature without any cutting element that provides trabeculorhexis on advancement of the shaft 6 in a direction of arrow A.

The distal end region of the shaft 6 can additionally or alternatively incorporate a disruptor 75 on the radially outward surface 8 that can provide debridement of the outer wall of Schlemm's canal on retraction in the direction opposite of arrow A. The disruptor 75 on the radially outward surface 8 can be a cutting element formed by one, two, or more discontinuities or windows 87 in the tubular shaft 6. FIG. 3A is a side view of the distal end region of the shaft 6 showing two windows 87 on the radially outward surface 8 just proximal to a location of the seam ripper 85 on the radially inward surface 7. FIG. 3B is a distal end perspective view of the radially outward surface 8 of the distal end region of the shaft 6 illustrating the configuration of the windows 87. The windows 87 can be formed by four edges cut into the radially outward surface 8 of the distal end region of the shaft 6 creating a leading edge 86, a trailing edge 88, and two lateral edges 90. The trailing edge 88 of the windows 87 can be blunt to avoid cutting tissue upon advancement in the direction of arrow A (see FIG. 3A). The leading edge 86 can be sharp to cut tissue upon retraction in the direction opposite of arrow A. The leading edge 86 of each window 87 need not be sharpened to debride the outer wall of Schlemm's as discussed elsewhere herein, for example, and can incorporate tines configured to project outward away from the long axis of the shaft 6 that flex inward toward the window 87 during advancement of the shaft as the shaft 6 and to flex outward away from the window 87 during retraction of the shaft 6 to catch on tissue thereby forming a one-way tissue disruptor. As with other implementations, the shaft 6 can be laser-cut to form the windows 87 and the seam ripper 85 and the probe tip 15 and any of the various features of the tissue disruptor. The shaft 6 can additionally be electropolished to improve sliding of the shaft 6 relative to the tissue.

The shaft 6 need not be tubular or even circular or hemi-cylindrical in cross-section. FIGS. 4A-4C, 5A-5C, 6A-6I, FIGS. 7A-7C, FIGS. 8A-8B, FIGS. 9A-9B each illustrate implementations of a tissue disruptor 10 on a shaft 6 formed from a flat sheet of material. FIG. 4A is a cross-sectional partial view of the device 2 showing the shaft 6 extending out from the distal end of the introducer tube 17 and forming a distal portion 11. The shaft 6 forming the distal portion 11 has radially outward surfaces of the shaft 6, which face away from the center of the central plane CP, and the radially inward side of the shaft 6, which faces toward the center of the central plane CP. FIG. 4B is a detail distal end perspective view of the distal end region of the shaft 6 showing the tissue engager 10. The distal end region of the shaft 6 can include a radially inward surface 7 connected to a radially outward surface 8 by two lateral sides 9. The distal end region of the shaft 6 can be curved or straight as described elsewhere herein. The distal end 73 of the shaft 6, which can have rounded distal-facing edges formed to a smooth ball tip that is blunt for atraumatic, is configured for smooth canal non-cutting and non-snagging goniotraction. The rounded edges on the distal face of the shaft 6 allow for the shaft 6 to slide better relative to the tissue compared to a tool with square edges. The lateral edges can be rounded or square cut. A disruptor 75 can be formed on the radially inward surface 7 of the distal portion 11 of the shaft 6 forming a seam ripper 85. The seam ripper 85 may project away from the shaft forming an angle C relative to the longitudinal axis A of the shaft 6. The distal facing surfaces of the disruptor 75, including the distal guide member 15 and the seam ripper 85, can be blunt without any cutting element.

The seam ripper 85 on the radially inward surface 7 can be a blunt tissue-engaging feature without any cutting element that provides trabeculorhexis on advancement of the shaft 6 in a direction of arrow A (see FIGS. 4B-4C). The distal end region of the shaft 6 can additionally or alternatively incorporate a disruptor 75 on the radially outward surface 8 that can provide debridement of the outer wall of Schlemm's canal on retraction in the direction opposite of arrow A. The disruptor 75 on the radially outward surface 8 can be a cutting element formed by one, two, or more teeth or tines 92 projecting radially outward from the longitudinal axis A of the shaft 6 or formed on a radially outward surface of the distal end region of the shaft 6. FIG. 4B is a side perspective view of the distal end region of the shaft 6 showing two tines 92 on the radially outward surface 8 just proximal to a location of the seam ripper 85 on the radially inward surface 7. The leading surface 94 of each of the tines 92 (i.e., the distal-facing surface) is smooth to slide along the outer wall of Schlemm's Canal during advancement of the shaft 6 in the direction of arrow A without causing tissue disruption or damage (see FIG. 4B-1). The trailing surface 96 of each of the tines 92 (i.e., the proximal-facing surface) is sharp or serrated or otherwise roughened to catch, cut and/or debride the outer wall of Schlemm's as the shaft is retracted in the direction opposite of arrow A causing tissue disruption. This arrangement of the tines 92 forms a one-way tissue disruptor that only alters the tissue of the outer wall during retraction of the shaft 6.

Schlemm's Canal is shaped at an angular orientation to the plane of the iris. The shaft 6 can be tilted relative to the plane of the iris, rather than perpendicular, to improve its conformation to the shape of Schlemm's Canal. For example, the shaft 6 can be tilted by about 30 degrees from perpendicular such that an anterior side of the shaft 6 is positioned further radially inward than a posterior side of the shaft 6. This provides better sliding motion of the shaft 6 relative to the anterior angle, better dilation of Schlemm's Canal and, a better modification of the outer wall of the canal by the disrupting elements on the radially outward surface of the shaft 6.

The shaft 6 can be laser-cut to form the seam ripper 85 on the radially inward surface 7, tines 92 on the radially outward surface 8, and other features of the disruptor 75. The laser-cut shaft 6 can be shape-set prior to or after cutting to achieve a curved distal portion 11. The seam ripper 85 can be located a distance proximal to the distal-most tip 15 of the shaft 6 providing a lead length of about 3 mm before tissue disruption due to the seam ripper 85 occurs. The diameter of the ripper tip 81 can be about 0.10 mm-0.25 mm, preferably about 0.15 mm. The diameter of the probe tip 73 can be about 0.15 mm-0.30 mm, preferably about 0.18 mm.

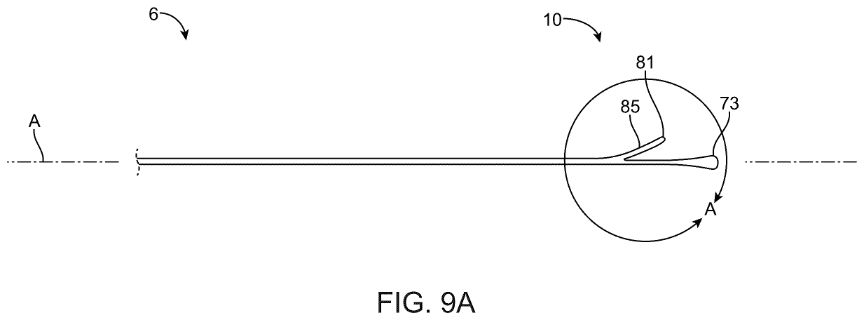

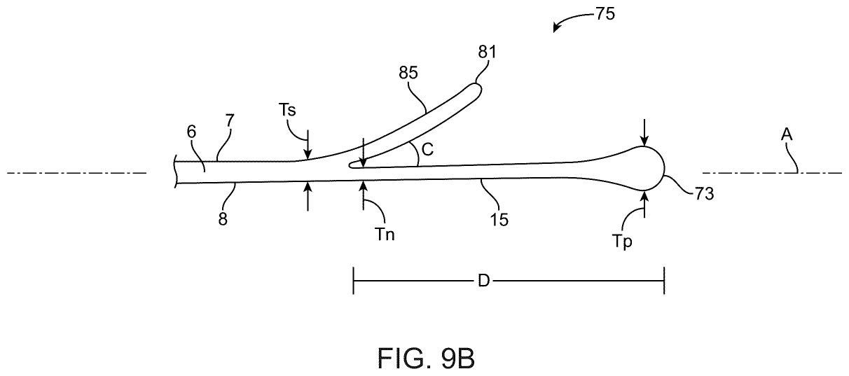

The shaft 6 can be formed of a highly flexible material that does not involve shape-setting. For example, the laser-cut shaft 6 may be straight and need not be shape-set into a curve at its distal portion 11, such as shown in FIGS. 9A, 12A, 13A, and 14A, which will be described in more detail below. A straight shaft 6 can be formed of a super-elastic materials, such as Nitinol, or non-super-elastic materials, such as stainless steel or plastic. The non-superelastic materials need not have the same resistance to deformation when retracted inside the introducer tube 17 as super-elastic materials. For example, the device can incorporate a non-superelastic shaft 6 packaged such that the distal end region of the shaft 6 remains extended beyond the distal end of the introducer tube 17. During use, the shaft 6 can be retracted and extended by the surgeon without significant degradation/compression of the features of the shaft 6 before it is discarded. The shaft 6 may undergo just one or two cycles of extension and retraction during use before being discarded or may be configured to undergo many cycles of extension and retraction before being discarded. The super-elastic shaft 6 may be able to undergo more extension cycles compared to a shaft made of stainless steel or polyimide film, which may undergo fewer extension cycles before needing to be discarded.

FIG. 9B is a detail view of FIG. 9A taken at circle A and illustrates thicknesses between the radially inward surface 7 and the radially outward surface 8 of the shaft 6 along a length of the distal end region of the shaft 6. These thicknesses are controlled by laser-cutting the flat stock sheet of material, such as Nitinol, to desired shapes. Other dimensions, such as a width across the radially inward surface 7 and corresponding width across the radially outward surface 8, are controlled by the thickness of the flat stock sheet. Thus, the width of the shaft 6 across the longitudinal axis A can be substantially the same along the entire length of the shaft 6. The width is preferably uniform along the entire length such that the shaft 6 appears uniformly rectangular from a top-down perspective. In contrast, the thickness of the shaft 6 between the radially inward surface 7 and the radially outward surface 8 can vary such that the shaft 6 takes on any of a variety of shapes from a lateral side view to provide the tissue disrupting geometry. The thickness of the shaft 6 between the radially inward surface 7 and the radially outward surface 8 at a location proximal to the region of the disruptor 75 is indicated in FIG. 9B as Ts. The thickness of the shaft 6 between the radially inward surface 7 and the radially outward surface 8 within the region of the disruptor 75, such as distal to a location where the tissue disruptor projects away from the radially inward surface of the shaft 6, that is also proximal to the probe tip 73 is indicated as Tn or the thickness of the neck region. A maximum thickness of the probe tip 73 between the radially inward surface 7 and the radially outward surface 8 is indicated in FIG. 9B as Tp. The nominal thicknesses and the thicknesses of these regions relative to one another enable the device to smoothly and reliably advance along at least about 90 degrees of Schlemm's Canal without snagging.

As discussed elsewhere herein, the shaft 6 can be formed by laser cutting the desired shape out of flat sheet of material. The thickness of the material sheet can vary. In some implementations, the material is preferably about 120-150 microns thick. If the sheet is thicker than 150 microns, the shaft 6 formed of the sheet can be too stiff for smooth advancement along the eye. In some implementations, the sheet forming the shaft 6 has a first thickness that is polished to a second thickness that is less than the first thickness. As such, the starting material of the shaft may be greater than this range and then polished down to having a thickness Ts that is within the range of 100-150 microns, preferably about 120-140 microns. The starting thickness of the material of the shaft 6 is selected to provide resistant to buckling during use of the disruptor to stay in-plane.

Still with regard to FIG. 9B, the maximum thickness Tp of the probe tip 73 is preferably greater than the thickness Ts of the distal end region of the shaft 6 and the thickness Tn of the neck region. The thickness Tn of the neck region is also less than the thickness Ts of the distal end region of the shaft 6. The maximum thickness Tp of the probe tip 73 is greater than the thickness Ts of the shaft 6 and preferably more than about 180 microns and less than about 360 microns. In some implementations, the maximum thickness Tp of the probe tip 73 is at least about 190 microns, at least about 200 microns, at least about 210 microns, at least about 220 microns, at least about 230 microns, at least about 240 microns, at least about 250 microns up to about less than 360 microns. If the shaft 6 has a thickness Ts between the radially inward surface 7 and the radially outward surface 8 that is about 100 microns, the maximum thickness Tp of the probe tip 73 between the radially inward surface 7 and radially outward surface 8 is greater than 100 microns (i.e., the thickness Ts of the shaft 6) by more than about 50% and up to about 3 times as large. The shaft 6 can be cut from a 100-micron to 150-micron thick nitinol sheet into a desired shape to create the disruptor 75 and seam ripper 85. The cut shape can be polished after cutting such that the corners and edges have a radius, such as about 30-micron radii. The rounded edges reduces resistance of the shaft 6, including the disruptor 75 and seam ripper 85, as the shaft 6 moves along the eye tissue. The probe tip 73 is enlarged in at least one dimension compared to the corresponding dimension of the shaft 6 (e.g., Ts=120 microns and Tp=240 microns). The maximum thickness Tp of the probe tip 73 can be thicker than the thickness Ts of the shaft 6 by at least 50%, by at least 60%, by at least 70%, by at least 80%, by at least 90%, by at least about 100% thicker than the thickness of the shaft 6 up to a nominal thickness of about 400 microns, or more preferably greater than about 180 microns and less than about 360 microns. The distal end of the probe tip 73 can have an enlarged feature or element has a width and is fully rounded such that it has a bulbous shape. The width of the rounded enlarged feature at the tip 73 is at least about 0.12 mm, at least about 0.15 mm, at least about 0.18 mm, at least about 0.20 mm, at least about 0.22 mm, at least about 0.24 mm, up to about 0.30 mm. The width of the rounded bulbous shape of the tip 73 may be described herein as a radius of curvature or a radius of the bulb.

The thickness Ts of the shaft 6 can be about 100-120 microns. The thickness Tn of the shaft 6 within the neck region can be less than about 100 microns down to about 50 microns, or about 55-95 microns, or about 60-80 microns. The maximum thickness Tp of the probe tip 73 can be anywhere about 120-400 microns, preferably greater than about 180 microns and less than about 360 microns. The thickness Tn of the shaft 6 within the region of the disruptor 75 and proximal to the probe tip 73 (i.e., the neck region) is preferably less than the maximum thickness Tp of the probe tip and also less than the thickness Ts of the shaft 6 proximal to the disruptor 75. The thickness Tn of the neck region can be about 30%-60% less than the thickness Tn of the shaft 6 proximal of the disruptor 75 and about 60%-75% less than the maximum thickness Tp of the probe tip 73.

Where the shape of the probe tip 73 is described as bulbous or ball shaped, it should be appreciated that the ball shape may only be within a certain 2-dimensional plane where the shape has a diameter across that is the maximum thickness Tp described above. The probe tip 73 shape and thickness relative to the thickness of the shaft 6 proximal to the probe tip 73 provides a self-guiding type of trackability along the canal. The shaft 6 can have a flexible column strength for guided forward disruption of the trabecular meshwork for continuous, non-morcellating tissue rhexis to disinsert or unroof the inner canal wall. The nominal values of the shaft thickness Ts, the neck thickness Tn, and the probe tip maximum thickness Tp can be selected to achieve a smooth advancing disruptor that avoids snagging on tissue during advancement around the canal. The wire material and polish level as well as the overall shape and curvature of the distal end region can be designed together with the nominal thickness values of these regions to avoid snagging and encourage advancement.

The seam ripper 85 can be formed to have an angle C relative to the longitudinal axis A of the shaft 6 and a length D as described elsewhere herein. The angle C of the seam ripper 85 can be about 20 to about 80 degrees, preferably about 30 to about 60 degrees. The length D of the seam ripper 85 can be about 1.50 mm to about 3.0 mm, preferably about 1.75 mm.

Again with respect to FIGS. 4A-4C, the tines 92 on the radially outward surface can be located proximal to the distal-most tip of the shaft 6, for example, the first tine 92 can be located about 3-4 mm proximal of the distal-most tip of the probe 15. The spacing between a trailing surface 96 of the first tine 92 to a trailing surface 96 of the second tine 92, etc. can be about 15 degrees along the distal portion or about 3 mm distance peak to peak. The angle formed between the trailing surface 96 of each tine 92 and the outer surface of the shaft 6 can be about 70 degrees, or between 45-90 degrees. Each tine 92 can have a height relative to the outer surface of the shaft 6. The height of the tines 92 creates a maximum thickness to the shaft 6 that is preferably sized to clear the inner diameter of the introducer tube 17 so that the shaft 6 including the tissue disruptor 10 at the distal end of the distal portion 11 can be fully retracted within the lumen 19 of the tube 17, for example, about 0.50 mm to about 0.80 mm, preferably about 0.67 mm. In some implementations, the maximum thickness of the shaft 6 for the tissue disruptor 10 can be at least about 0.320 mm to about 0.525 mm, or about 0.345 mm to about 0.350 mm. Table 1 lists example dimensions of various implementations of shafts 6 considered herein.

TABLE 1

| Diameter | Thickness of | Maximum | Thickness of | Length |
|---|---|---|---|---|
| of ball tip | shaft distal to | thickness of | shaft proximal | of |
| (Tp in | disruptor | disruptor at | to disruptor | probe tip |
| mm) | (Tn in mm) | apex (mm) | (Ts in mm) | (mm) |
| 0.24 | 0.080 | 0.500 | 0.10 | 1.70 |
| 0.24 | 0.060 | 0.345 | 0.10 | 1.75 |
| 0.24 | 0.060 | 0.345 | 0.08 | 1.75 |
| 0.24 | 0.060 | 0.375 | 0.10 | 1.51 |
| 0.24 | 0.065 | 0.500 | 0.09 | 1.70 |
| 0.24 | 0.065 | 0.500 | 0.09 | 1.70 |

The shaft 6 can be laser-cut, shape-set, and/or electropolished as described elsewhere herein. The amount of electropolish is preferably about 25%. Thus, if the stock material for the shaft 6 is about 0.0270 mm thick, electropolishing results in about 0.0230 mm, or removal of about 15 μm of material.

FIGS. 1C-1D, 4A-4C, 5A-5C, 6A-6I, 7A-7C, 8A-8B, 9A-9B illustrate implementations of a shaft 6 formed from a Nitinol flat sheet. The sheet can have a thickness of about 100 microns to about 150 microns, preferably 100 microns to 120 microns. The shaft 6 can be used with any of the devices described herein and extend through a lumen 19 of an introducer tube 17 projecting from a distal end region of the housing 13 where the tube 17 is straight and extends along a long axis from its proximal end to its distal end or is at least partially curved along its length including a dual curve as discussed above.

The sheet (having a thickness of about 100-150 microns, for example, or as large as 2000 microns) may be cut to a width of about 0.010 cm to about 0.015 cm and having a total length about 150 cm up to about 170 cm, the distal portion 11 being about 20 mm of the total length. The sheet may be laser-shaped into the elongate, flexible shaft having a non-circular cross-section and a distal end region comprising any of a variety of tissue disrupting profiles, which will be described in more detail below. The distal portion 11 of the shaft 6 may be shaped into a curve forming a curved distal portion 11 having a central plane CP as described elsewhere herein (e.g., about 220 degrees-230 degrees) and as shown in FIGS. 1A, 1C-1D, 2A, 2C, 4A, 5A-5B, 6A, 7A, and 8A. The curved distal portion 11 can provide tracking around the trabecular meshwork with a minimum amount of effort or resistance. The distal portion 11 also can be straight, which will be described in more detail below.

FIG. 5B shows a detail view of a distal portion 11 of the distal end region of the shaft 6 having a tissue engager 10 of FIG. 5A. The distal end region of the shaft 6 has a curve with a radially inward surface 7 connected to a radially outward surface 8 by two lateral sides 9. FIG. 5C shows the distal end 73 of the shaft 6, which can have rounded distal-facing edges formed to have a smooth ball tip that is blunt for circumferential gonio-traction. The rounded edges on the distal face of the shaft 6 allow for the shaft to slide better relative to the tissue compared to square edges. The lateral edges can be rounded or square cut. A disruptor 75 is formed on the inner surface of the distal portion 11 of the shaft 6. The distal face 76 of the disruptor 75 may slope gently from the thickness of the shaft 6, the thickness being between the inward and outward surfaces of the shaft distal to the disruptor 75, to the maximum thickness of the disruptor 75, the maximum thickness being between the inward and outward surfaces of the shaft. The proximal face 77 of the disruptor 75 can taper down to the thickness of the shaft 6, the thickness being between the inward and outward surfaces of the shaft proximal to the disruptor 75. The distal face 76 of the disruptor is a blunt tissue-engaging surface without any cutting element. A short segment forming a distal guide member 15 can extend distal to the location of the disruptor 75. The length of the distal guide member 15 can vary, such as from about 1 mm up to about 5 mm (preferably about 1.50 mm to about 1.75 mm). Thus, the smooth ball tip of the shaft can be located this same distance away from the distal face of the disruptor.

The thickness of the shaft between the inward and outward surfaces distal to the disruptor can be about 100-150 microns. The thickness of the shaft between the inward and outward surfaces proximal to the disruptor can also be about 100-150 microns. The thickness of the shaft proximal to the disruptor can be larger (e.g., as large as up to about 2000 microns) because once the disruptor has opened the canal, the proximal shaft dimensions are no longer limited by the size of the canal, but by the size of the corneal incision. The thickness of the shaft distal to the disruptor can also be larger than about 150 microns, such as up to about 450 microns. The maximum thickness of the tissue disruptor between the inward and outward surfaces can be about 250-325 microns or 250-600 microns (e.g., about 450-600 microns, preferably at least about 550 microns). In some implementations, the maximum thickness of the tissue disruptor between the inward 7 and outward surfaces 8 can be at least about 320 microns to about 525 microns, or about 345 microns to about 350 microns. The tissue disruptor can be a fixed dilatory segment of the shaft configured to dilate and stretch Schlemm's canal prior to and during modification and/or disruption of the inner and/or outer walls of Schlemm's canal. Stretching of the canal wall may further improve outflow as it may expand the canaliculi and ostia.

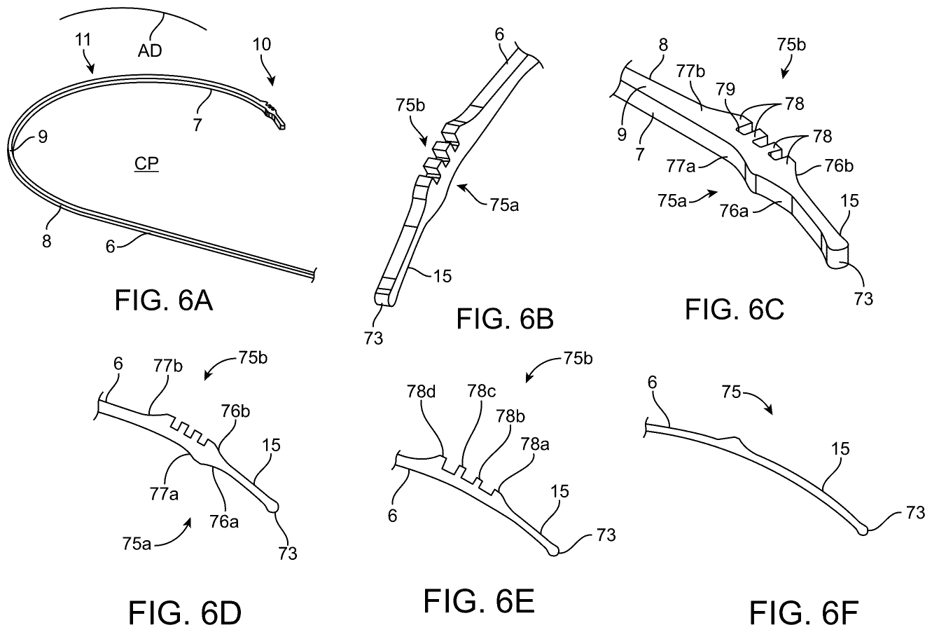

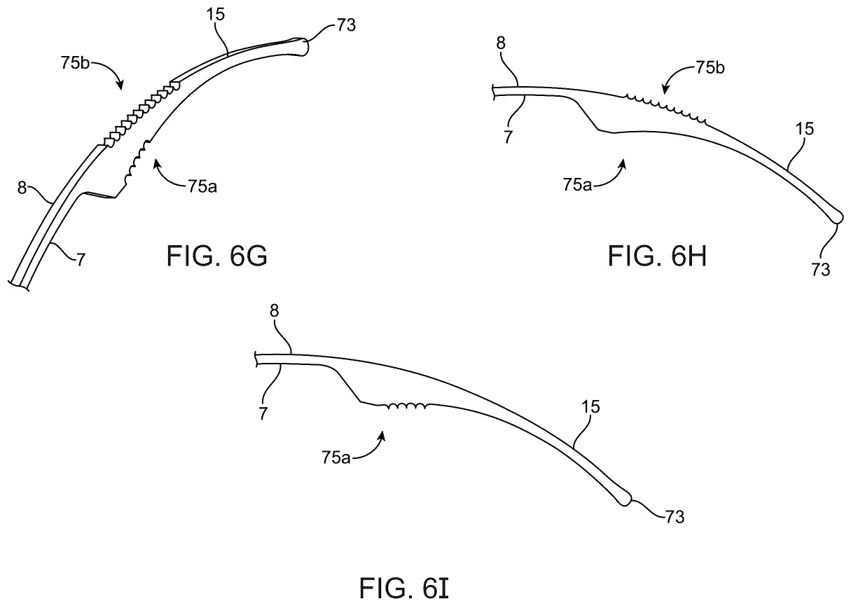

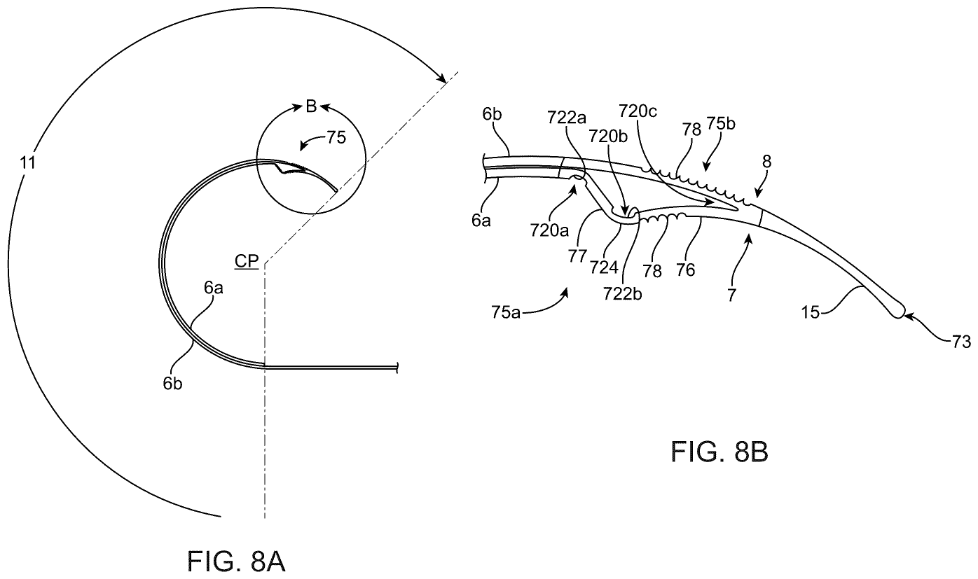

The elongate, flexible shaft of superelastic material is sized and configured for ab interno insertion into the anterior chamber of the eye. The distal end region of the shaft can be shaped into a curve having a central plane or can be straight. The cross-sectional shape of the shaft, if taken transverse to the length of the shaft between a distal end and a proximal end, can be generally non-circular, such as square or rectangular. As mentioned above, the distal end region of the shaft can include a radially inward surface 7 connected to a radially outward surface 8 by two lateral sides 9. The inward surface 7 and outward surface 8 of the shaft 6 along the distal portion can be curved whereas the two lateral sides 9 can be planar or straight. In other implementations, the distal portion is straight such that the inward surface 7, the outward surface 8, and the two lateral sides 9 are each substantially planar (see FIG. 9A). The tissue engager 10 of the shaft 6 shown in FIGS. 5A-5C has a single inwardly projecting tissue disruptor 75 on the radially inward surface 7 of the shaft. FIGS. 6A-6C and also FIGS. 8A-8B, FIGS. 12A-12C, 13A-13C, and 14A-14B illustrate other implementations of a tissue engager 10 on the shaft 6 having an inwardly projecting disruptor 75a on the radially inward surface 7 of the distal end region of the shaft and an outwardly projecting toothed disruptor 75b on the radially outward surface 8 opposite the inwardly projecting disruptor. The disruptors on the inward and outward surfaces can be opposite one another along the length of the shaft or on opposite sides by off-set longitudinally along the length of the shaft. For example, the outer wall disrupting feature may be further proximal than the inner wall disrupting feature located further distal along the shaft so that the inner wall and outer wall disrupting features are on opposite sides, but not perfectly opposite one another and merely near one another. As with other implementations, the shaft 6 can be formed from a Nitinol flat sheet having a thickness of about 100 microns to about 500 microns, preferably about 100-150 microns, more preferably about 100-120 microns, cut to a desired specification. The flat sheet can be cut into an elongate, flexible shaft (e.g., 150-170 cm) having a non-circular cross-section and a distal end region having a particular tissue disrupting profile. A proximal end region of the shaft can be cut to have a width of about 0.010 cm to about 0.015 cm. A distal end region of the shaft can be cut to have a tissue disruptor profile that widens relative to the proximal end region of the shaft. The tissue disrupting profile can vary and can include a tissue disruptor 75 located proximal of a distal-most end 73 of the shaft 6 on at least one of the inner surface 7 and the outer surface 8. As discussed elsewhere herein, the tissue disruptor 75 can include a distal face, a proximal face, and a maximum thickness between the inward and outward surfaces. The distal face may slope from a first thickness of the shaft 6 distal to the tissue disruptor 75 towards the maximum thickness. The proximal face may taper down from the maximum thickness to a second thickness of the shaft 6 proximal to the tissue disruptor 75. The thickness of the shaft 6 between the lateral sides 9 is a function of the thickness of the flat sheet stock material. Although the sheet stock material thickness can vary as is available in the art, preferably a 100-micron thick Nitinol sheet up to about a 150 micron thick Nitinol sheet is desired. The thicknesses (and overall shape profile) of the shaft 6 between the inward and outward surfaces can vary and is controlled by the programmed cut profile. Different cut profiles may be selected based on the intended anatomy of the tool and the type of tissue disruption desired. The maximum thickness of the tissue disruptor 75 can vary but is preferably about 250-325 microns when measured between the inward and outward surfaces or at least about 250 microns, at least about 320 microns, at least about 325 microns, at least about 345 microns, at least about 350 microns, up to about 525 microns compared to the thickness of the shaft proximal and distal to the tissue disruptor, which is about 100-150 microns. The thickness of the shaft between the inward and outward surfaces proximal to the disruptor can be larger than this up to about 2000 microns whereas the thickness of the shaft between the inward and outward surfaces distal to the disruptor can be 100-450 microns The thickness of the shaft between the inward and outward surfaces distal to the disruptor can be thinner than the thickness of the shaft proximal of the disruptor, for example, about 55 microns to about 100 microns, preferably about 80 microns. The tissue disrupting profile at the distal end region of the shaft 6 may include two tissue disruptors-one formed on the inner surface 7 and one formed on the outer surface 8 opposite the first tissue disruptor or even at a different location along a length of the shaft 6. The manufacturing methods of laser-shaping a flat sheet of super-elastic memory-shape material into a dimension (e.g., between 5-5000 microns) allows for an assembly-free manner of creating micro-interventional tools for use in Schlemm's canal or near an anterior angle of the eye.

Again, with respect to FIGS. 6A-6C, the distal end region of the shaft 6 may be shaped to have a distal portion 11 that has a central plane CP as described elsewhere herein. The central plane CP can be due to a shape-set curve in the shaft 6 or the distal end region of the shaft 6 can be substantially straight and take on a curved configuration having a central plane CP during use in an eye by virtue of its flexibility conforming to the curvature of Schlemm's canal. The distal-most end 73 of the shaft 6 may be blunt or formed (such as by laser-cutting) into a smooth ball tip. The edges on the distal face of the shaft 6 forming the distal-most end 73 are preferable rounded to allow for the shaft to slide better relative to the tissue compared to square edges. The lateral edges (i.e., edges formed where the inner surface 7 meets the lateral sides 9 and the outer surface 8 meet the lateral sides 9) can be rounded or square-cut. The disruptor 75a, proximal of the distal-most end 73, formed on the inner surface may slope gently from the thickness of the shaft 6 distal to the disruptor 75a to a thickness of the disruptor 75a. The proximal face 77a of the inwardly facing disruptor 75a can taper down from the thickness of the disruptor 75a to a thickness of the shaft 6 proximal to the disruptor 75a. A distal guide member 15 can extend distal to the location of the disruptor 75a. A distal face 76a of the inwardly facing disruptor 75a slopes from the thickness of the guide member 15 up to a maximum thickness of the disruptor 75a. The length of the distal guide member 15 can vary, such as from about 1 mm and 5 mm, between about 1 mm and 3 mm, about 1.5 mm to about 2 mm, or less than 3 mm down to about 1 mm, preferably about 1.50 mm-1.75 mm.

The shaft 6 can be used with any of the devices described herein and extend through a lumen 19 of an introducer tube 17 projecting from a distal end region of the housing 13 where the tube 17 is straight and extends along a long axis from its proximal end to its distal end or is at least partially curved along its length including a dual curve as discussed above.

As mentioned, the tissue engager 10 of the shaft 6 can have an additional disruptor 75b projecting from the outer surface 8. Still regarding FIGS. 6A-6C and also FIGS. 6D-6I, the disruptor 75b formed on the outer surface 8 of the shaft 6 can incorporate tines like those described above that form a plurality of teeth 78. The distal guide member 15 extending distal to the disruptor 75b may slope gently towards the first tooth 78 of the disruptor 75b. A proximal face 77b of the outwardly facing disruptor 75b can taper down from the thickness of the disruptor 75b to a thickness of the shaft 6 proximal to the disruptor 75b. A distal face 76b of the disruptor 75b slopes from the thickness of the guide member 15 (e.g., about 100-150 microns) up to a thickness of the first tooth 78 (e.g., about 250 microns). The teeth 78 of the disruptor 75b can be separated from one another by a gap 79. The gap 79 may vary in size so that the teeth 78 are spaced from one another by about 200 microns up to about 350 microns. The gap 79 of the device of FIG. 6D is about 100 microns whereas the gap 79 of the device in FIG. 6E is about 210 microns resulting in narrower teeth 78 compared to the device of FIG. 6D. Each tooth 78 can have the same height (see FIG. 6D) or one or more of the plurality of teeth 78 may vary in height (see FIG. 6E). In the implementation shown in FIG. 6E, a first tooth 78a projects outward a first height so that its thickness (i.e., the thickness from inner surface to outer surface of the shaft at that location) is about 250 microns, a second tooth 78b projects outward a second height so that its thickness is about 275 microns, a third tooth 78c projects outward a third height so that its thickness is about 300 microns, and a fourth tooth 78d projects outward a fourth height so that its thickness is the maximum thickness of the disruptor 75b (e.g., 325 microns). The plurality of teeth 78 of the disruptor 75b need not vary in the height they project outward and can be more uniform or alternating in heights. Any of a variety of combinations is considered herein. The geometry of each tooth 78 can be generally square-cut although other geometries are considered.

The geometry of each tooth 78 may be more triangular forming a plurality of shearing serrations (see FIGS. 6G-6I). The serrated teeth 78 of the disruptor 75 shown in FIG. 6G are positioned on both the inner surface 7 and the outer surface 8 of the shaft 6 whereas the serrated teeth 78 of the disruptor 75 shown in FIG. 6H are only on the outer surface 8. FIG. 6I shows a single-sided disruptor 75 with serrated teeth 78 on the inner surface 7. The disruptor 75 can have a combination of square-cut and triangular cut teeth 78 or serrations like shown in FIG. 6G-6H. The teeth 78 in the disruptor 75 of FIG. 6I are only on the inner surface 7. Any of a variety of geometries is considered herein on both the inward and outward surfaces 7, 8 or on just the inner surface 7 or just the outer surface 8 of the shaft 6. The maximum thickness of the disruptor 75, regardless the shape or configuration of the teeth or serrations, can be between 550-600 microns to ensure the tissue is not merely stretched. The maximum thickness of the disruptor can be about 320 microns to about 525 microns, or about 345 microns to about 500 microns.

FIG. 6D shows a distal end region of a shaft 6 with a disruptor 75a on the inner surface 7 as well as the toothed disruptor 75b on the outer surface 8. FIG. 6E shows a distal end region of a shaft 6 with a disruptor 75b on only the outer surface 8. The presence of the two disruptors on the device of FIG. 6D creates an initial ramp height that is about 330 microns thick compared to the initial ramp height of FIG. 6E that is only about 250 microns thick.

In some implementations, the disruptor 75 of the shaft 6 can project radially outward from the outer surface 8 of the shaft 6 and yet disrupt tissues positioned radially inward relative to the shaft 6. FIG. 6F illustrates an implementation of a tissue engager 10 of the shaft 6 having a disruptor 75 projecting radially outward from the outer surface 8 of the shaft 6 and the inner surface 7 being relatively uniform or smooth with the curve proximal and distal to the location of the disruptor 75. The outwardly projecting disruptor 75 of FIG. 6F may disrupt tissues radially inward relative to the shaft 6 (i.e., the trabecular meshwork) during advancement due to the disruptor 75 abutting against the outer wall and being sized and shaped to urge the shaft 6 in a radially inward direction so that the inner curvature of the shaft 6, which may be uniform and smooth without any disruptor per se, drags along the trabecular meshwork thereby disrupting it. The shape of the outer curvature disruptor 75 can be designed to slide along the outer wall to direct the distal tip 73 of the shaft inward back out of the canal to disrupt the trabecular meshwork during advancement of the shaft 6 in the advancement direction.

The shearing serrations or teeth 78 positioned on a wedge-shaped disruptor 75 can include a canal-probing dilating ball-tipped distal end 73 extending distal to the disruptor 75. The ball-tipped distal end 73 can allow guided traction within the canal during forward disruption. As discussed elsewhere herein, the cross-sectional shape of the shaft 6, if taken transverse to the length of the shaft 6 between a distal end and a proximal end, can be generally non-circular, such as square or rectangular. The shaft 6 if not cut into a ball-shape or other atraumatic shape would be problematic during advancement through the canal because the leading square edges would tend to snag or cut tissue. The forward-facing edges of the shaft 6 at the distal end 73 are cut to be rounded to avoid this.

The length of the guide member 15 separating the ball tip 73 from the wedge disruptor 75 can vary, but may be between 1 mm and 5 mm, between about 1 mm and 3 mm, about 1.5 mm to about 2 mm, or less than 3 mm down to about 1 mm, preferably about 1.50 mm-1.75 mm.

In some implementations, the device 2 can include a distal probe 15 projecting distally from the tissue engager 10 (see FIGS. 2D, 2E, 4B-4C, 5C, 6A-6I, 8A-8B, 9A-9B, 12A-12C, 13A-13C, and 14A-14B). The probe 15 can be integral with or formed by the distal end of the shaft 6. For example, the tissue engager 10 can be a region of the shaft 6 that is proximal to the distal-most end 73 of the shaft 6 such that the distal probe 15 is formed by the segment of the shaft 6 that extends distal to the tissue engager 10. The probe 15 can serve as a probing canal engagement front end and can be blunt and non-incisional. The probe 15 can guide the device along Schlemm's canal as the shaft 6 is advanced and the tissue engager disrupts the trabecular meshwork. The probe 15 can insert within Schlemm's Canal prior to disruption of the trabecular meshwork as the tissue engager 10 is advanced along the angle. The probe 15 can have an outer diameter that is about 100-280 microns. The shaft 6 can have an outer diameter that is about 100-800 microns. The probe 15 can be designed for intracanalicular placement/deployment and guided traction along the canal while the shaft 6 can be designed to advance the probe 15 while remaining outside the canal.

The probe 15 can be elongate so that at least a portion of the device 2 inserts within Schlemm's Canal prior to the tissue engager 10 disrupting the trabecular meshwork and eliminating the canal. For example, the probe 15 can extend distally from the tissue engager 10 by 300 to 5000 microns, or by 30 microns to 500 microns, although the probe 15 may be shorter or longer. The probe 15 may be a piece of formed sheet metal and extend distally from the tissue engager 10 by 30-500 microns. The probe 15 also can be very short so that substantially no portion of the device 2 inserts within Schlemm's Canal prior to the tissue engager 10 disrupting the trabecular meshwork and eliminating the canal. The device 2 may also have no distal, probe 15 that inserts within Schlemm's Canal such that the tissue engager 10 essentially disinserts or scrapes away the trabecular meshwork without any entry of Schlemm's by the device 2. Thus, the tissue engager 10 need not be fully or even partially inserted through the trabecular meshwork tissue and within the Schlemm's Canal, such as with a distal probe 15, to disrupt tissue.

Described herein are disruptors designed to flex inward to a narrower configuration, such as for delivery into the eye within an introducer 17, and to flex outward to a wider configuration useful during disruption. The disruptor can have an unbiased, resting configuration that has an expanded dimension, such as upon extension outside the introducer 17, and is configured to be biased into a collapsed, smaller dimension, such as upon retraction inside the introducer 17. The expandable/collapsible disruptors can be designed to repeated move between their expanded and collapsed states without deformation. The disruptors of FIGS. 1C-1D, 2A-2G, 3A-3C, 4A-4C, 7A-7C, 8A-8B, 9A-9B, 12A-12C, 13A-13C, and 14A-14B can each incorporate an element that is capable of being flexed into a narrower configuration for delivery into the eye outward to a wider configuration upon advancing outside the introducer, such as during disruption. The seam ripper 85 in FIGS. 2D, 3A, 3C, 4B, 7A-7C, 9A-9B, and others is configured to flex towards the radially inward surface of the probe 15 such that angle C between the seam ripper 85 and the distal probe 15 is more acute. The seam ripper 85 is also configured to return to its resting shape and flex outward away from the radially inward surface of the probe 156 such that the angle C between the seam ripper 85 and the distal probe 15 is less acute.

FIGS. 8A-8B illustrate an implementation of a shaft 6 incorporating a disruptor 75 configured to move between a collapsed configuration and an expanded configuration. FIGS. 9A-9B, 12A-12C, 13A-13C, and 14A-14B illustrate similar implementations of a shaft 6 incorporating a collapsible/expandable disruptor 75. As with other implementations described herein, the distal end region of the shaft 6 can incorporate a distal portion 11 that is curved and defines the plane of curvature in use that is aligned generally with the plane on which Schlemm's Canal lies (see FIGS. 8A-8B). The shaft 6 may also incorporate a distal portion 11 that is substantially straight (see FIGS. 9A-9B, 12A-12C, 13A-13C, and 14A-14B). The disruptor 75 can project from the inner surface 7 and/or the outer surface 8 of the shaft 6. When in the collapsed configuration, the disruptor 75 can be sized to fit within an inner diameter of the introducer 17. When in the expanded configuration, the disruptor 75 can be sized larger than the inner diameter of the introducer 17.

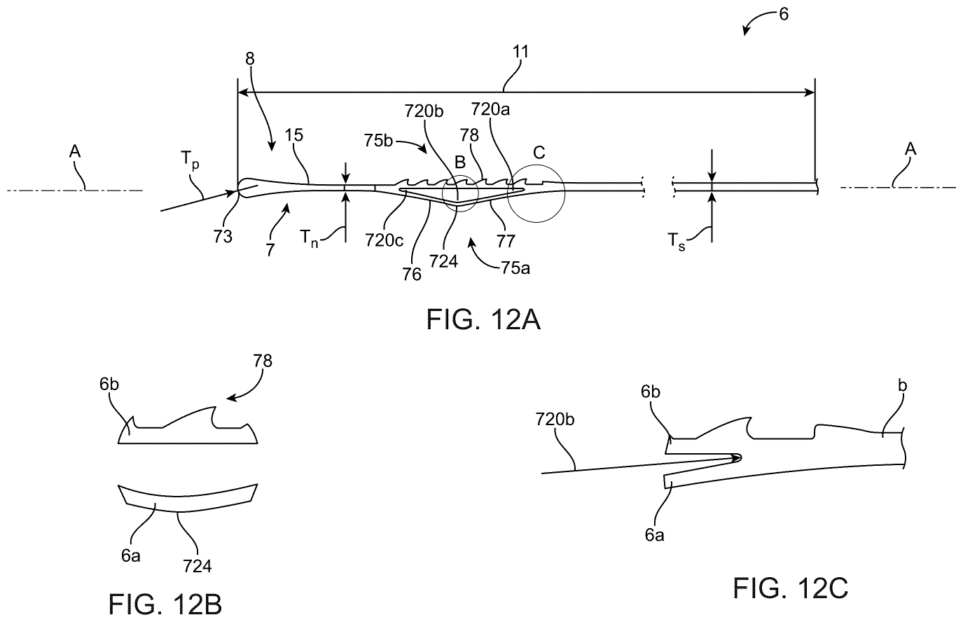

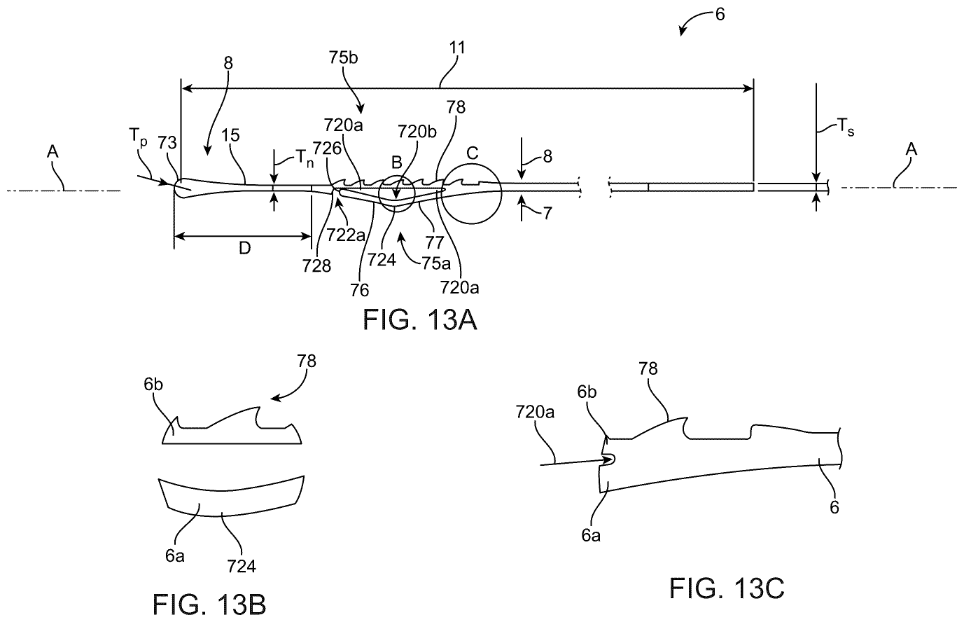

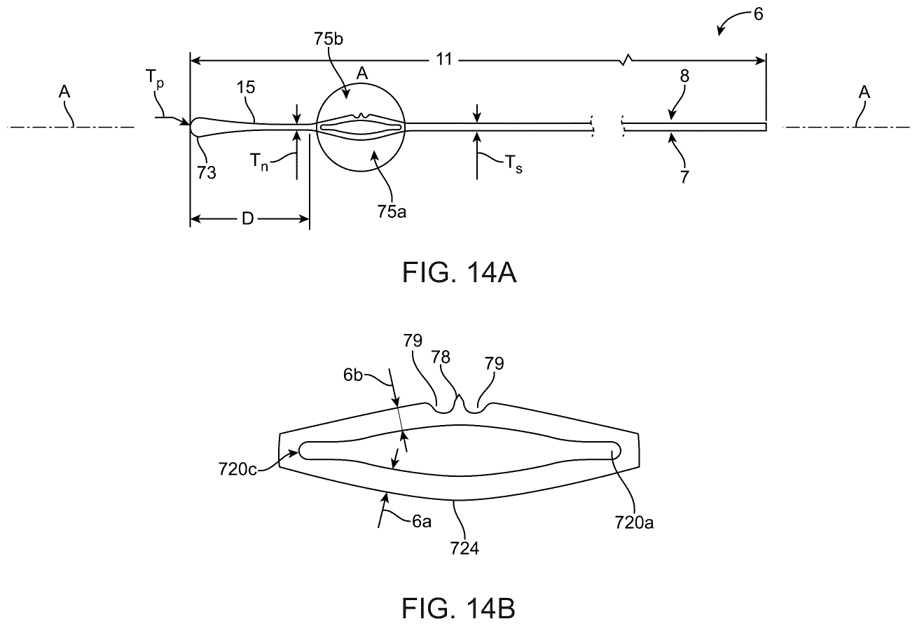

FIG. 8A shows the distal end region of the shaft 6 and FIG. 8B is a detail view of FIG. 8A taken at circle B. The distal end region of the shaft 6 in FIG. 8A is shape-set to achieve a curve. FIG. 9A shows the distal end region of the shaft 6 and FIG. 9B is a detail view of FIG. 9A taken at circle A. FIG. 12A shows the distal end region of the shaft 6 and FIGS. 12B and 12C are detail views of FIG. 12A taken at circles B and C, respectively. FIG. 13A shows the distal end region of the shaft 6 and FIGS. 13B and 13C are detail views of FIG. 13A taken at circles B and C, respectively. FIG. 14A shows the distal end region of the shaft 6 and FIG. 14B is a detail view of FIG. 14A taken at circle A. The distal end region of the shaft 6 in FIGS. 9A, 12A, 13A, and 14A re not shape-set to achieve a curve and are instead substantially straight.

In one or more of these embodiments, the distal end region of the shaft forming the distal portion 11 can include two shaft segments 6a, 6b. A first shaft segment 6a lies on an inside of the distal portion 11 and a second shaft segment 6b lies on an outside of the distal portion 11. At least one of the shaft segments 6a, 6b extends proximally to form a proximal end region of the shaft 6. The shaft segment 6b on the outside of the curve extends to form the proximal end region of the shaft 6 and the shaft segment 6a on the inside of the curve extends no further proximal than a location of the distal portion 11. The shaft segments 6a, 6b can be separate from one another along at least a length of the distal portion 11 to a location just distal to the disruptor 75 forming the distal guide member 15. There, the shaft segments 6a, 6b become one. Or, in different words, the distal guide member 15 splits into two shaft segments 6a, 6b at a location of the disruptor 75, the two shaft segments 6a, 6b extending along at least a portion of the distal portion 11. In at least the implementation shown in FIG. 8A, the distal portion 11 is curved, including the distal-most tip 73 to a more proximal region of the shaft 6 where it becomes substantially straight, can extend at least 220 degrees up to about 230 degrees.

Still with respect to FIGS. 8A-8B, 12A-12C, 13A-13C, and 14A-14B, the shaft can incorporate two disruptors-one disruptor 75a on a radially inward side of the distal portion 11 and one disruptor 75b on a radially outward side of the distal portion 11. The first shaft segment 6a on an inside of the distal portion 11 can form the disruptor 75a that projects radially inward. The second shaft segment 6b lying on an outside of the distal portion 11 can form or incorporate the disruptor 75b that projects radially outward. When the disruptor 75a on the inside is in the expanded configuration, the first shaft segment 6a projects inward away from the second shaft segment 6b forming a triangular-shaped space between the shaft segments 6a, 6b. The shaft segments 6a, 6b can incorporate one or more flex regions 720 (see FIGS. 8B, 12A-12C, 13A-13C, 14A-14B). A first flex region 720a can be located near a proximal end of the disruptor 75a and a second flex region 720b can be located near an apex 724 of the disruptor 75a. A third flex region 720c can be located near a distal end of the disruptor 75a. When in the expanded configuration, the first shaft segment 6a relaxes away from the second shaft segment 6b in a radially inward direction increasing an angle between the shaft segments 6a, 6b at the first flex region 720a and third flex region 720c while decreasing the angle at the second flex region 720b. A first cut-out 722a can be formed on a radially inner surface of the first shaft segment 6a, the cut-out 722a forming a first area of decreased thickness in the shaft segment 6a. A second cut-out 722b can be formed on a radially outer surface of the first shaft segment 6a forming a second area of decreased thickness along the shaft segment 6a. When the first shaft segment 6a flexes away from the second shaft segment 6b during expansion of the disruptor 75a, an angle of the first shaft segment 6a at the first flex region 720a increases because the regions on either side of the cut-out 722a bend toward one another. An angle of the first shaft segment 6a at the third flex region 720c increases as well. An angle of the first shaft segment 6a at the second flex region 720b decreases as the regions on either side of the cut-out 722b bend toward one another. A distal face 76 of the disruptor 75a slopes gently radially inward moving towards the apex 724 of the disruptor 75a until reaching a maximum height of the apex 724 and thus, maximum thickness of the shaft 6. A proximal face 77 of the disruptor 75a slopes gently back in a radially outward direction (e.g., towards a thickness of the shaft 6 just proximal of the disruptor 75a, which can have a thickness of the two shaft portions 6a, 6b together). The inside radius of the proximal and distal flex regions 720 can vary from about 0.10 mm to about 0.05 mm, or about 0.013 mm to about 0.03 mm. The radius of the proximal flex region 720 in the implementation shown in FIGS. 12A and 13A can be about 0.013 mm and the radius of the proximal flex region 720 in the implementation shown in FIG. 14A can be about 0.03 mm.

In some implementations, the shaft segments 6a, 6b can be formed of a flat stock (e.g., Nitinol or stainless steel or plastic) having a thickness of about 100-150 microns. Thus, the thickness of the two shaft portions 6a, 6b together can be about 200-300 microns or no greater than about 450 microns so that when the disruptor 75a is in the collapsed configuration the shaft 6 fits entirely within the lumen of the introducer 17. The maximum outer diameter of the shaft 6 between the outer-most surface of the second shaft segment 6b and an innermost surface of the first shaft segment 6a at the apex 724 of the disruptor 75a when in the expanded configuration can be greater than about 450 microns up to about 600 microns, or about 320 microns to about 525 microns, or about 345 microns to about 500 microns.

The first and second shaft segments 6a, 6b can be in contact with each other such that when the disruptor 75a is in the collapsed configuration the thickness of the shaft 6 is minimized. The first and second shaft segments 6a, 6b can remain unattached from one another proximal of the disruptor 75a to accommodate the change in angle at the flex regions 720. This allows for the first shaft segment 6a to slide relative to the second shaft segment 6b during expansion of the disruptor 75a. The first shaft segment 6a can slide distally along the second shaft segment 6b to allow for the radially inward projection of the shaft segment 6a during expansion of the disruptor 75a. The first shaft segment 6a can also slide proximally along the second shaft segment 6b to allow for collapse of the first shaft segment 6a during collapse of the disruptor 75a.

FIGS. 13A-13C illustrate an implementation of a disruptor 75a having a first flex region 720a formed by a cut-out 722a on a radially inner surface of the first shaft segment 6a. Rather than forming a first area of decreased thickness in the shaft segment 6a as in the embodiment of FIG. 8A, the cut-out 722a extends through the full thickness of the first shaft segment 6a such that a discontinuity in the shaft segment 6a is formed. This allows the distal face 76 of the disruptor 75a to slide relative to the second shaft segment 6b to reduce the height of the apex 724. The disruptor 75a can be constrained in its collapsed configuration upon retracting the shaft 6 inside the introducer tube 17. As the shaft 6 is retracted and the proximal face 77 abuts against the distal end of the introducer tube 17 surrounding the opening 29, a distal-facing end 726 of the first shaft segment 6a at the cut-out 722a slides distally toward a proximal-facing end 728 of the first shaft segment 6a at the cut-out 722a thereby narrowing the gap between the ends 726, 728 and reducing the angle at the flex region 720 and reducing the height of the apex 724. The distal-facing end 726 of the first shaft segment 6a at the cut-out 722a can also slide proximally away from the proximal-facing end 728 of the first shaft segment 6a at the cut-out 722a thereby widening the gap between the ends 726, 728 and increasing the angle at the flex region 720, such as upon extending the shaft 6 outside the introducer tube 17 releasing the disruptor 75a from its constraint. The disruptor 75a relaxes back into its expanded configuration increasing the height of the apex 724.

In the implementation of FIGS. 12A-12C and 13A-13C, the first shaft segment 6a forming the disruptor 75a is movable relative to the second shaft segment 6b forming the disruptor 75b, which remains substantially static and substantially parallel with the longitudinal axis A of the shaft 6. FIGS. 14A-14B illustrate another implementation of a shaft 6 having a first disruptor 75a formed by a first shaft segment 6a and a second disruptor 75b formed by a second shaft segment 6b with flex regions 720 connecting them. One or both of the shaft segments 6a, 6b can be movable. The first disruptor 75a can be configured to bow away from the longitudinal axis A of the shaft 6, such as in a radially inward direction. The second disruptor 75b can be configured to bow away from the longitudinal axis A of the shaft 6, such as in a radially outward direction. Each of the first and second disruptors 75a, 75b can move relative to one another to transition from a collapsed, smaller outer dimension configuration to an expanded, larger outer dimension configuration.

Still with respect to FIGS. 14A-4B, the first shaft segment 6a lying on the inside of the distal portion 11 that forms the disruptor 75a on the radially inward side of the distal portion 11 is smoothly curved compared to the more angular bend of the shaft segment 6a in the shafts 6 shown in FIGS. 12A-12C or 13A-13C. A flex region 720a is located near a proximal end of the disruptor 75a and a flex region 720c is located near a distal end of the disruptor 75a with each of the shaft segments 6a, 6b spanning between the flex regions 720a, 720c. One or both of the shaft segments 6a, 6b can bow outward between the flex regions 720a, 720c forming a maximum thickness at a midpoint between the flex regions 720a, 720c. The shaft segments 6a, 6b can become flatter upon retraction of the shaft 6 within the lumen of the introducer tube 17 and can become rounder upon extension of the shaft 6 from the lumen of the introducer tube 17. The inside radius of the proximal and distal flex regions 720 can be about 0.03 mm.

Although the figures illustrating the flex regions show disruptors on both the radially inward and radially outward sides of the shaft, it should be appreciated that the shaft having the flexible/collapsible disruptor need have only a single side that is configured to disrupt tissue. As such, the shaft designed with a disruptor having one or more flex regions can disrupt via a feature on a radially inward side, a feature on the radially outward side, or features on both radially inward and radially outward.

Again with respect to FIGS. 8A-8B, 12A-12C, 13A-13C, and 14A-14B, the distal-most end or probe tip 73 of the shaft 6 (i.e., the distal end of the probe 15) can be shaped into a smooth ball tip that is blunt for insertion through trabecular meshwork tissue and into a portion of Schlemm's canal for smooth canal non-cutting and non-snagging goniotraction as described elsewhere herein. The distal face 76 of the disruptor 75a can be a smooth, tissue-engaging surface without any cutting element (see FIGS. 12A, 13A, 14A-14B) or can incorporate one or more tines or teeth or other surface feature 78 like those shown in FIG. 8B.

The distal end region of the shaft 6 can incorporate an additional disruptor 75b projecting from the outer surface 8 (see FIGS. 8A-8B, 12A-12C, 13A-13C, and 14A-14B). The disruptor 75b on the outer surface 8 can incorporate one or more tines or teeth or other surface feature 78 configured to snag tissue of an outer wall of Schlemm's. The geometry of the surface features 78, whether on the inner or outer surface, can be sharp or generally square-cut or other geometries described herein. The surface features 78 can project above a surface of the shaft segment 6a, 6b by at least about 20 microns.

The disruptor 75b shown in FIGS. 12A-12C and 13A-13C can incorporate a plurality of tines or teeth as discussed elsewhere herein. The disruptor 75b shown in FIGS. 14A-14B can incorporate a single tine or tooth 78 formed by a pair of cut-outs or gaps 79 on the radially outer surface 8 of the shaft 6. The geometry of the tooth 78 can vary, but is generally substantially sharp to disrupt the outer wall of Schlemm's Canal as the disruptor 75b engages the tissue, as discussed elsewhere herein. The maximum height between the outer-most end of the tooth 78 on the second shaft segment 6b and the maximally bowed surface of the first shaft segment 6a can be about 350 microns to about 500 microns. The collapsible element 75b of FIG. 14A-14B is designed such that it can be retracted inside the introducer tube 17 without permanently deforming the disruptor 75b. When the disruptor 75b is extended from the introducer tube 17 it can expand to the desired disrupting shape. The tooth 78 on the second shaft segment 6b is positioned to bear against the outer wall of Schlemm's canal as the shaft 6 is advanced around the canal thereby scoring, cutting, and/or debriding the outer canal wall. When retracted, the disruptor 75b collapses again as it is withdrawn into the introducer tube 17. Also, as discussed elsewhere herein, the shaft 6 can be shape-set to form a curved distal portion 11 as shown in FIGS. 1A, 1C-1D, 2A, 2C, 4A, 5A-5B, 6A, 7A, and 8A. As the shaft 6 extends outside the introducer tube 17, the distal portion 11 curves thereby redirecting the tissue engager 10 relative to the introducer tube 17. The shaft 6 need not curve and can be straight as shown in FIGS. 9A, 12A, 13A, and 14A. The straight distal portion 11 provides easy and smooth advancement around the trabecular meshwork, particularly where the shaft 6 incorporates thinner segments. The dimensions and geometry of the tip, thumb, and neck, whether the distal portion 11 is curved or straight, can provide a flexibility to the distal portion 11 that allows the shaft 6 to adjust and conform to the variances in diameter and other anatomical feature of Schlemm's Canal, Trabecular meshwork, and unique dimensions of a patient's eye, including Canals that are deeper or “out of plane”. A distal portion 11 that is straight can be introduced into Schlemm's Canal via a less acutely angled introducer tube 17 compared to a distal portion 11 that is curved. This provides greater flexibility for a surgeon to decide where to enter Schlemm's Canal. The straight shaft 6 can be disposed outwardly toward the back wall of Schlemm's Canal while it transits resulting in less likelihood for unintentional escape/exit of the canal during use that can lead to inadvertent creation of a suprachoroidal cleft or iris damage. The straight shaft 6 that is formed to be highly flexible and conforming can allow for a certain amount of self-direction when a surgeon does not maintain a consistent orientation during advancement of the shaft. The shaft 6 can engage with the trabecular meshwork and act like an alignment rail. The bulbous tip effectively surfs around the canal regardless of the orientation of the handpiece. This can also occur with the curved wire, but to a lesser degree.

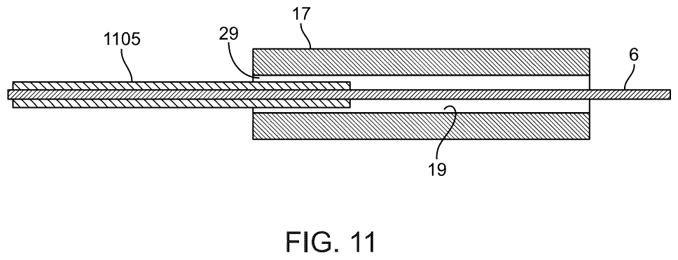

As discussed elsewhere herein, the material used to form the shaft 6 can be less than 120 microns, such as about 100 microns down to about 80 microns or less. Shafts 6 having these thin segments can buckle during advancement along a segment of the eye, particularly if the exposed length of the shaft 6 outside the introducer tube 17 is very long. As discussed elsewhere herein, the shaft 6 is designed to extend a distance beyond a distal end of the introducer tube 17. The exposed length of the shaft 6, including the tissue disrupting portion of the distal end region, is highly flexible yet also has some columnar strength to disrupt tissues, such as the trabecular meshwork and/or the outer wall of Schlemm's Canal. Folds, constrictions, and inconsistent surfaces of the Canal can lead to buckling of the shaft 6 having low columnar strength. For example, where the thickness of the material sheet used to form the shaft 6 is less than 120 microns for greater flexibility, the columnar strength is reduced. The devices described herein can incorporate additional support features to constrain the exposed region of the flexible, low columnar strength shaft 6 to prevent buckling of the shaft 6 during advancement through eye tissue. FIG. 11 illustrates a stiffening sleeve 1105 surrounding at least a portion of the shaft 6 for support purposes. The left side of FIG. 11 is more proximal and the right side of FIG. 11 is more distal illustrating a portion of the shaft 6 extending distal to the introducer tube 17. The figure is shown in schematic and none of the specifics of the instrument are illustrated. The stiffening sleeve 1105 can be fixed to an external surface of the shaft 6 along at least a proximal region of the shaft 6 leaving exposed a more distal region of the shaft 6. The stiffening sleeve 1105 and the shaft 6 can be advanced and retracted together through the lumen 19 of the introducer tube 17 so that the more distal region of the shaft 6 can exit the introducer tube 17 while the proximal region of the shaft 6 stiffened by the sleeve 1105 remains inside the introducer lumen 19. The sleeve 1105 has a stiffness that functions to reinforce at least the proximal region of the shaft 6 as the more distal region of the shaft 6 moves through the eye tissue thereby reducing the potential for the shaft 6 to buckle. The stiffening sleeve 1105 surrounds the shaft 6 along its length such that the sleeve 1105 remains inside the introducer tube 17 proximal of the distal curve. Where the shaft 6 is sufficiently flexible to advance through the distal curve 23 of the introducer tube 17 and sufficiently flexible to conform to the curvature of the eye upon advancement through the canal, the sleeve 1105 is sufficiently inflexible. As such, the sleeve 1105 preferably remains proximal of the curve at the distal end region 23 of the introducer tube 17 even upon full extension of the shaft 6 relative to the introducer tube 17.

The tissue engager 10 can disrupt the trabecular meshwork from the anterior chamber angle as it is advanced around the eye without entering the canal. Alternatively, the tissue engager 10 can disrupt the sclera following removal or disinsertion of the trabecular meshwork. In other words, because the Schlemm's Canal has already had one of its walls disrupted (i.e., the inner wall), there is no “canal” to be cannulated or catheterized. An open channel has been formed revealing the scleral wall ab interno so that the tissue may be engaged by one or more features of the devices described herein. Thus, the ab interno method can involve a “non-canal” or “outside the canal” sort of gonio-intervention or modification of the anterior angle of the eye. This non-catheterized, non-cannulated access to the scleral wall in the anterior angle provides a greater flexibility in the sort of interventions that can be performed because there is no need for canal catheterization. The tools for the intervention described herein can be larger than tools that are required to fit within the Schlemm's Canal between the trabecular meshwork and the scleral wall, but still sufficiently small for ab interno insertion through a self-sealing corneal incision or puncture. “Catheterize” refers to entering Schlemm's canal for greater than 4 clock hours.

The various surfaces and dimensions described herein for all implementations shall be defined by the view associated with particular surface or orientation. When considering a rectangular-shaped cross-section each of four defined sides may be well defined. When a circular cross-sectional shape is used, it is understood that the definition of upper surface and lower surface would subdivide the circular cross-section into two half circles. Similarly, the lateral walls would subdivide into two half circles which means that each part of the surface may define two surfaces since the surfaces are exposed in two orientations and contribute to both width and height.

The tissue engager 10 can include a disruptor on a radially-inward side of the shaft 6 and/or a disruptor on a radially-outward side of the shaft 6. The radially-outward disruptor can be a cutting element, debrider, or another feature on the outer-facing surface. In some implementations, the disruptor is the cutting element configured to cut a circumferential slit in the canal outer wall as the device is advanced. The tissue engager 10 may simultaneously gather trabecular tissue with the disruptor 75 on the radially-inward surface so that the tissue stretches and tears as described herein and cut scleral tissue with the cutting element. The device 2 may also operate without trabeculorhexis and may be practiced with the radially outward cutting or debridement only. The device 2 may also operate to perform a first method step to disrupt an inner wall of Schlemm's Canal (i.e., the trabecular meshwork) with a disruptor and as a second method step perform modification of the outer wall (i.e., sclera) with a cutting element on a radially outer surface. Thus, the tissue modifications of the inner and outer walls of Schlemm's canal can be performed simultaneously with a cutting element and a disruptor or as sequential steps with the different tools. Tissue modification of the inner wall can occur without modifying the outer wall. Tissue modification of the outer wall can occur without modifying the inner wall. The cutting element may also be referred to herein as a tissue disruptor on the radially outward surface of the shaft 6.

The disruptors of the implementations of FIGS. 5A-5C, 6A-6I, 8A-8B, 12A-12C, 13A-13C, and 14A-14B, can also incorporate asymmetric disruption that achieves blunt disruption only along a radially inward direction as the shaft 6 is advanced along the anterior angle and achieves cutting-type disruption only along a radially outward direction as the shaft 6 is retracted from the anterior angle. Thus, the disruptor on the radially outward surface does not cut as the shaft 6 is advanced and only cuts as the shaft is withdrawn.

In some implementations, the cutting element on the radially outward surface can form a continuous cut, slit, gouge, shaven region, or other type of cut region in the outer wall of Schlemm's canal to increase an effective size of Schlemm's canal. The effective size is increased since the cut increases the potential enclosed volume of the canal. Any length of cut may be formed, and the device is capable of forming a continuous cut through at least 45 degrees, and may be at least 90 degrees, of Schlemm's canal in use. The shaft 6 is also capable of developing the spring response described herein that may also provide advantages when advancing the cutting element on the radially outward surface into the canal wall. The cutting element on the radially outward surface can be used to modify not just the outside wall of Schlemm's canal, but anywhere along a band of the eye extending from the ciliary body to the limbus depending on a rotational angle of the tissue engager 10.

The cutting element on the radially outward surface can be used to form an elongate (in the circumferential direction) cut or debridment that increases the available surface area available for fluid transfer. The cut also effectively shortens the fluid path since the fluid path is generally radially outward and the cut is formed generally in a radially outward direction. The tools described herein may be also practiced without removing the trabecular meshwork in a canaloplasty procedure. The tissue engager and cutting element can be reduced in size and delivered through a cannula to form one or more circumferential cuts in the radially outer (sclera) wall. The elongate cut may provide improvement in fluid flow as a primary canaloplasty therapy for the reasons discussed above. The devices described herein can be capable of performing trabeculorhexis without cutting. The devices described herein can also incorporate a cutting element. The device described herein can incorporate one or more features that rip/strip/tear the tissue. For example, all aspects of the shaft 6 may be practiced with the tissue engager 10 cutting tissue.

The device 2 can include features designed to modify the gonio scleral wall after and/or prior to removal/disruption/excision of Schlemm's canal that can include the cutting element on the radially outward surface or other surface modifying elements on a surface of the shaft 6 or tissue engager 10 that is directed radially outward, including one or more blades, abrasive surfaces, thinning elements, or other structural modifiers of the gonio wall of the eye. The devices described herein need not canal catheterization to access the scleral wall in the angle.

The device 2 can be coupled to a source of suction so that aspiration and/or infusion of fluids can be performed through the lumen 19 of the introducer tube 17. Alternatively, tissue and fluids may be removed/delivered using a separate suction device in fluid communication with the lumen 19.

The devices described herein can modify the outer wall of Schlemm's Canal, such as by scraping, debriding, cutting the tissue using a feature on the shaft 6 that bears against the outer wall. This outer wall modification can enhance outflow from the eye and lower intraocular pressure while the trabecular meshwork remains spared. The outer wall modification can also be associated with inner wall modifications so that a single pass within Schlemm's Canal can modify both the inner and outer walls simultaneously.

Any of the devices described herein can incorporate a shaft that is shape-set into a curve at its distal end region or is not shape-set and thus, remains substantially straight at its distal end region. The straight shaft can have geometries similar to those described for the curved shafts, but may be used with introducer tubes that are downsized slightly compared to the curved shaft. The shaft used with the downsized introducer tube may thus have dimensions that are slightly reduced compared to the shaft used with larger introducer tube.

All the devices described herein can be designed so that the trabecular meshwork is not torn or disrupted in any way except for the location where the shaft 6 enters Schlemm's Canal. This approach of disrupting the outer wall can provide control of intraocular pressure while sparing the trabecular meshwork. The shafts 6 described herein, regardless their specific dimensions and geometry, can be designed to collapse as the shaft 6 is retracted into the introducer tube 17 and, alternatively, so it can expand to its clinically functional shape when extended from the introducer tube 17. The shaft 6 can include any of a variety of features on the radially outward portion of the shaft 6 to engage the outer wall of Schlemm's canal, such as to cut or score or otherwise modify the outer wall, during advancement of the shaft 6 through the Canal, retraction of the shaft 6 through the Canal, and/or both advancement and retraction. The exact geometry of the feature can vary.

### Methods

Use of the devices 2 is now described. The device 2 is introduced into the eye ab interno using any suitable approach. A corneal incision or puncture can be formed and the distal end of the device 2 inserted through the opening. The elongate shaft 6 can be retracted at least partially so that the tissue engager 10 on the distal portion 11 of the elongate shaft 6 is positioned, preferably, inside the lumen 19 of the introducer tube 17. An entry opening and a first terminal opening can be formed through the trabecular meshwork to access Schlemm's canal. The entry opening can be formed using a conventional bladed instrument. The introducer tube 17 of the device 2 can then be introduced into the entry opening and the tissue engager 10 advanced toward the first terminal opening by extending the shaft 6 distally from the housing 13. As the tissue engager 10 is advanced, the flexible shaft 6 changes the orientation of tissue engager 10 to conform the lower surface 8 of the tissue engager 10 radially outward towards the outer wall (i.e., the scleral wall) and the upper surface 7 radially inward towards the center of the central plane CP. In this manner, the user may not be required to substantially change the orientation or position of the housing 13 as the tissue engager 10 is advanced. Depending on the configuration of the shaft 6 tissue engager 10, only the trabecular meshwork is disrupted during extension of the shaft 6, or both the trabecular meshwork and the outer wall of Schlemm's Canal is disrupted during extension of the shaft 6, or only the outer wall of Schlemm's Canal is disrupted during extension of the shaft 6, or the trabecular meshwork is disrupted during extension of the shaft 6 and the outer wall of Schlemm's Canal is disrupted during withdrawal of the shaft 6, or the outer wall of Schlemm's Canal is disrupted during extension of the shaft 6 and the trabecular meshwork is disrupted during withdrawal of the shaft 6.

In some implementations, when the tissue engager 10 reaches the first terminal opening, a first portion of tissue has been disrupted to expose the scleral wall to the anterior chamber. The device 2 may be used to disrupt another portion of the trabecular meshwork to expose more of the scleral wall by forming a second terminal opening and advancing the tissue engager 10 to the second terminal opening. The entry opening is created by removing or incising the trabecular meshwork to the outer wall of Schlemm's canal or through Schlemm's canal to expose the sclera. The trabecular meshwork disrupted and released by the present devices may also be parted off with a separate device or with the devices themselves (by cutting or tearing) as described.

As used herein, the term “displace tissue” or “disrupt tissue” includes both blunt engagement to move the tissue but also cutting the tissue to move the tissue in the path of the tissue engager. The terms “gather” tissue and “gathering” tissue means that tissue collects and bunches up in front of or at the tissue engager. The gathered tissue may be somewhat compressed as it collects ahead of the device. Displacement of this gathered tissue advantageously rips/tears/shears the tissue along both lateral sides without cutting at both lateral sides so that a strip of material is being freed from the native tissue. Use of a cutting element may result in a slit being formed without meaningful removal of material. Use of a cutting element may also result in a trough, gouge, or channel being formed with some removal of material. Use of a rounded tube or element may result in simply tearing the trabecular meshwork open along a seam without meaningful removing material. The ability of the devices described herein to gather tissue does not require the device to gather all of the tissue being removed. The gathered tissue may slide to one side or the other or “over” the tissue engager so that the tissue engager gathering a different part of the trabecular meshwork and tearing/ripping tissue free by displacing the newly gathered different part of the trabecular meshwork. The device can gather tissue corresponding to the width of the tissue engaging element while a rounded tube (or a cutting element) is not capable of gathering tissue in this manner.

The advancing direction as used herein is defined as a local vector that is essentially a tangent to the circular shape of the Schlemm's canal. As such, the advancing direction essentially follows the curvature of the Schlemm's canal and can incorporate more than a single direction. All compatible features of any embodiment shall be interchangeable with any other embodiment and all such combinations are expressly incorporated herein.

In addition, the non-cutting probe and/or the tissue micro-disruptor/trabeculorhexis element may both have tissue modulating surface elements on their outer surface that can engage and/or modulate the surface of the external canal wall. For example, such elements may include micro-abrasive surface for canal wall cleaning, debridement and/or thinning. Further embodiments of a combined trabeculorhexis-canaloplasty device whereby in addition to the trabeculorhexis configuration, the device has features designed to change, modulate, abrade, shave, thin, micro-perforate the outer/external/contralateral-to-the-TM canal wall. This can be achieved by a modified surface architecture of the guide-probe and/or the tissue disruptor and/or the flexible shaft with abrasive non-smooth surface including but not limited to a grating configuration, notching and other surface elements designed to treat and modify the surface the canal wall surface during movement of the device along the contour of the canal. This combined trabeculorhexis-canaloplasty procedure will not only disinsert and remove the TM (trabecular meshwork), but also can improve and change the anatomy of the remaining canal wall for additional improvement of aqueous outflow. In addition, a further embodiment where the surface of such ab-interno device (guide-probe and tissue disruptor) can be coated with a hemostatic coating (e.g., silver nitrate) which can reduce bleeding during the procedure. The simultaneous modification of the inner and outer walls can be performed with a combined device.

The method of disrupting the inner and/or outer walls can also be a two-step method where a first step is performed to modify the trabecular meshwork, for example, with a first device and a second step is performed to modify the outer wall, for example, with a second device. The outer wall modification can occur after the trabecular meshwork modification. The outer wall modification occurring after the trabecular meshwork modification can be a function of the direction of motion with the tissue engager 10. As discussed elsewhere herein, the tissue engager 10 can incorporate a first disruptor (e.g., a radially inward side of the shaft 6) and a second disruptor (e.g., on a radially outward side of the shaft 6). The first disruptor is configured to disrupt one wall of Schlemm's canal (e.g., Trabecular meshwork) on forward motion (extension) of the shaft 6 and the second disruptor slides along the opposing wall of Schlemm's canal on forward motion without any disruption. The second disruptor is configured to disrupt the opposing wall of Schlemm's canal only upon rearward motion (withdrawal) of the shaft 6. Opposite walls of the canal can be disrupted asynchronously upon a single cycle of shaft extension and retraction to reduce friction and excess resistance. Resistance and fraction between the shaft 6 and the tissues of the canal can also be reduced by surface treatments of the materials, such as electropolish or other polishing processes that smooth traction in the canal.

The devices described herein are preferably introduced ab interno but may be practiced with ab externo approach. The device can be moved by advancing to tear tissue, the device may do so preferably without cutting or ablating the tissue. Cutting devices and even a cutting element with the devices may be provided.

The methods of ab interno circumferential continuous trabeculorhexis described herein can be performed alone without any implantable structure (including no implantable structures coupled to the housing) left in the eye, such as supraciliary interventions for suprachoroidal outflow. The ab interno circumferential continuous trabeculorhexis methods described herein can be performed in conjunction with implantation of a shunt or stent-like structure as the implant for dual-outflow interventions. The trabecular intervention can be circumferential goniotomy and the uveoscleral intervention can be bio-reinforced cyclodialysis with an implant configured to scaffold the space. The methods can also be performed in conjunction with standard phacoemulsification. In a preferred implementation, the implant can be part of a supraciliary interventional system for suprachoroidal outflow is a biological, cell-based or tissue-based material that provides biocompatible aqueous outflow enhancement. The bio-tissue reinforcement provided by the implant can provide improved tolerability and safety over non-biological, polymeric shunts known in the art. In an example implementation, a biologic tissue or biologically-derived material is harvested or generated in vitro and formed into an implant, also referred to herein as a biotissue stent. The implant may be an elongated body or material that has an internal lumen to provide a pathway for drainage. In a preferred implementation, the implant is an elongated body or strip of tissue that does not have an internal lumen and is configured to maintain a cleft and provide supraciliary stenting (or stenting within another anatomical location such as within Schlemm's Canal or trans-scleral). Lumen-based devices can be limited by the lumen acting as a tract for fibrotic occlusion. The implant formed from the tissue is implanted into the eye via an ab interno delivery pathway to provide aqueous outflow from the anterior chamber. The implant can be used as a phacoemulsification adjunct or stand-alone treatment to glaucoma as a micro-invasive glaucoma surgery (MIGS) treatment. The implant can be part of a dual-outflow intervention for Open Angle Glaucoma in which microinterventional canaloplasty tools described herein are used to increase trabecular outflow and a biotissue implant provides reinforcement at the ciliary cleft to enhance suprachoroidal outflow.

The biotissue implant can be implanted before or after Schlemm's canal intervention. The method can include performing ab interno continuous goniotomy and inner wall trabeculotomy (i.e., continuous circumferential trabeculorhexis, along a segment of a circumference of an eye. The segment can be greater than 90 degrees and up to about 180 degrees. The method can include positioning at least one implant within a ciliary cleft after performing the continuous goniotomy, the implant being a minimally-modified biological tissue, such as scleral, corneal, or amniotic membrane tissue. Visualization for positioning of the biotissue implant may be improved due to prior disruption of the trabecular meshwork.

Use of the terms like stent, implant, shunt, bio-tissue, or tissue is not intended to be limiting to any one structure or material. The structure implanted can but need not be a material that is absorbed substantially into the eye tissue after placement in the eye such that, once absorbed, a space may remain where the structure was previously located. The structure once implanted may also remain in place for an extended period and not substantially erode or absorb.

The stent can be made from biologically-derived material that does not cause toxic or injurious effects once implanted in a patient. The term “biologically-derived material” includes naturally-occurring biological materials and synthesized biological materials and combinations thereof that are suitable for implantation into the eye. Biologically-derived material includes a material that is a natural biostructure having a biological arrangement naturally found within a mammalian subject including organs or parts of organs formed of tissues, and tissues formed of materials grouped together according to structure and function. Biologically-derived material includes tissues such as corneal, scleral, or cartilaginous tissues as well as acellular biomatrix tissue. Biologically-derived material includes amniotic membrane. Tissues considered herein can include any of a variety of tissues including muscle, epithelial, connective, and nervous tissues. Biologically-derived material includes tissue harvested from a donor or the patient, organs, parts of organs, and tissues from a subject including a piece of tissue suitable for transplant including an autograft, allograft, and xenograft material. Biologically-derived material includes naturally-occurring biological material including any material naturally found in the body of a mammal. Biologically-derived material as used herein also includes material that is engineered to have a biological arrangement similar to a natural biostructure. For example, the material can be synthesized using in vitro techniques such as by seeding a three-dimensional scaffold or matrix with appropriate cells, engineered or 3D printing material to form a bio-construct suitable for implantation. Biologically-derived material as used herein also includes material that is cell-derived including stem cell(s)-derived material. In some implementations, the biologically-derived material includes an injectable hyaluronate hydrogels or viscomaterials such as GEL-ONE Cross-linked Hyaluronate (Zimmer).

Biologically-derived materials can include naturally-occurring biological tissue including any material naturally found in the body of a mammal that is minimally manipulated or more than minimally manipulated according to FDA guidance under 21 CFR § 1271.3 (f) such that the processing of the biological tissue does not alter the relevant biological characteristics of the tissue (see Regulatory Considerations for Human Cells, Tissues, and Cellular and Tissue-Based Products: Minimal Manipulation and Homologous Use, www.fda.gov/regulatory-information/search-fda-guidance-documents/regulatory-considerations-human-cells-tissues-and-cellular-and-tissue-based-products-minimal).

The biologically-derived material, sometimes referred to herein as bio-tissue or bio-material, which is used to form the implant can vary and can be, for example, corneal tissue, scleral tissue, amniotic membrane tissue, cartilaginous tissue, collagenous tissue, or other firm biologic tissue. The bio-tissue can be of hydrophilic or hydrophobic nature. The bio-tissue can include or be impregnated with one or more therapeutic agents for additional treatment of an eye disease process.

The biologically-derived material can include or be capable of releasing one or more factors of the biologically-derived material for providing additional treatment of a disease or condition. For example, the material can be a tissue that releases healing factors derived from the tissue that have anti-fibrotic, anti-inflammatory, anti-neovascular effects, or the like for repair and regeneration at the site of implantation or near the site of implantation. The material can be whole amniotic membrane that releases one or more regenerative and anti-fibrotic factors that aid in the control of inflammation and scarring including, but not limited to VEGF (Vascular Endothelial Growth Factor), VEGF-R (VEGF Receptor), ANG1 (Angiopoietin 1), TIMP-1 (Collagenase inhibitor), TIMP-2 (Collagenase Inhibitor), IL-1B (Interleukin 1B), PDGF-AA (Platelet Derived Growth Factor), TGFb3 (Transforming Growth Factor Beta 3), bFGF (Basic Fibroblast Growth Factor), and HGF (Heptocyte Growth Factor). The amniotic membrane contains both growth promoting and growth inhibiting proteins (see, e.g., Clinical Ophthalmology 2019:13, 887-894).

The amniotic membrane tissue can be derived from amniotic sac of the placenta. The tissue can be freeze-dried, lyophilized membrane or otherwise minimally manipulated prior to implantation (see, e.g., SURGRAFT dehydrated amniotic sheets, or SURSIGHT ocular amniotic membrane allograft, Surgenex Scottsdale, AZ).

The amniotic membrane can be used as an adjunct treatment with a biostent as an implantable bio-eluting scaffold. The amniotic membrane can also be used alone as a primary treatment. The biostent alone or with amniotic membrane provided as an adjunct treatment can be implanted in any of a variety of locations including the suprachoroidal space, supraciliary space, Schlemm's canal, cornea, anterior chamber, posterior chamber, intravitreal, epiretinal, subretinal, or other part of the eye.

The bio-stent material can be used in combination with one or more therapeutic agents such that it can be used to additionally deliver the agent to the eye. In an implementation, the bio-tissue can be embedded with slow-release pellets or soaked in a therapeutic agent for slow-release delivery to the target tissue.

Non-biologic material includes synthetic materials prepared through artificial synthesis, processing, or manufacture that may be biologically compatible, but that are not cell-based or tissue-based. For example, non-biologic material includes polymers, copolymers, polymer blends, and plastics. Non-biologic material includes inorganic polymers such as silicone rubber, polysiloxanes, polysilanes, and organic polymers such as polyethylene, polypropylene, polyvinyls, polyimide, etc.

The stent(s) implanted in the eye may have a structure and/or permeability that allows for aqueous outflow from the anterior chamber when positioned within a cyclodialysis cleft. The biologically-derived material can be minimally-modified or minimally-manipulated tissue for use in the eye. The minimally-modified biologically-derived material does not involve the combination of the material with another article although water, sterilizing, preserving, cryopreservatives, storage agent, and/or pharmaceutical or therapeutic agent(s), and the like can be included. The minimally-modified biologically-derived material does not have a systemic effect once implanted and is not dependent upon the metabolic activity of any living cells for its primary function. The biologically-derived material can be minimally-manipulated during each step of the method of preparation and use so that the original relevant characteristics of the biologic tissue are maintained.

As used herein, the terms are often used with reference to a view of the device in use and may be modified as described below to provide further clarification of these term. The term advancing direction may be modified with the term “which is oriented in a tangential direction with respect to the circular shape of the eye.” The term height may be modified with the term “which is radially oriented with respect to the circular shape of the eye”. Similarly, the term “width” may be modified with the term “which is oriented perpendicular to the advancing direction and the height” or with the term “oriented parallel to a central axis of the eye”. Finally, the terms upper or upper surface and lower or lower surface may be modified with the terms “which is oriented on a radially inward surface with respect to the circular shape of the eye” and “oriented on a radially outward surface with respect to the circular shape of the eye”, respectively. The above referenced terms apply to circular, tubular and frustoconical shapes equally.

Suitable materials or combinations of materials for the preparation of the various components of the devices disclosed herein suitable for ab interno interventions such as inner wall trabeculorhexis and/or outer wall canaloplasty are provided throughout. It should be appreciated that other suitable materials are considered. The interventional devices can be constructed from any implant grade material that can provide the functions required. Materials that may be employed in this device could be but are not limited to nylons, PVDF (polyvinylidene fluoride), PMMA (polymethyl methacrylate), polyimide, Nitinol, titanium, stainless steel, or other implant grade materials. The interventional devices may be made from a combination of materials that are geometrically mated together, chemically bonded or welded to one another, over-molded, encapsulated, or other means for joining multiple materials. A given device element may be made of multiple materials.

The elongate shaft may be formed of materials, such as titanium, stainless steel, or other metal or metal alloys, polyether ether ketone (PEEK), ceramics, rigid plastics, or other materials. The material of the shaft is relatively firm and has the structural ability to exert a force on the outer wall for modification of the outer wall using the disruptor projecting towards the outer wall. The outer wall modification can occur after prior goniotomy with another device or can occur in combination with the goniotomy disruptor using, for example, one or more of the non-catheterized disruptor tools described above.

In various implementations, description is made with reference to the figures. However, certain implementations may be practiced without one or more of these specific details, or in combination with other known methods and configurations. In the description, numerous specific details are set forth, such as specific configurations, dimensions, and processes, to provide a thorough understanding of the implementations. In other instances, well-known processes and manufacturing techniques have not been described detail to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various placed throughout this specification are not necessarily referring to the same embodiment or implementation. Furthermore, the particular features, structures, configurations, or characteristics may be combined in any suitable manner in one or more implementations.

The devices and systems described herein can incorporate any of a variety of features. Elements or features of one implementation of a device and system described herein can be incorporated alternatively or in combination with elements or features of another implementation of a device and system described herein. For the sake of brevity, explicit descriptions of each of those combinations may be omitted although the various combinations are to be considered herein. Additionally, the devices and systems described herein can be positioned in the eye and need not be implanted specifically as shown in the figures or as described herein. The various devices can be implanted, positioned and adjusted etc. according to a variety of different methods and using a variety of different devices and systems. The various devices can be adjusted before, during as well as any time after implantation. Provided are some representative descriptions of how the various devices may be implanted and positioned, however, for the sake of brevity explicit descriptions of each method with respect to each implant or system may be omitted.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “upper,” “lower,” “top”, “bottom,” “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value. One inch or 1″ corresponds to 2.54 cm (SI-units).

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

### P EMBODIMENTS

P Embodiment 1. A device for disrupting tissue in an eye, the device comprising a distal portion sized and configured for ab interno insertion into an anterior chamber of the eye. The distal portion comprises an elongate, flexible shaft of super-elastic memory-shape material. The shaft comprises a distal end region shaped into a curve having a central plane; a distal-most end; a first tissue disruptor proximal of the distal-most end formed on a radially inward surface of the curve of the distal end region; and a second tissue disruptor proximal of the distal-most end formed on a radially outward surface of the curve of the distal end region. During use, the distal-most end is configured to be inserted through trabecular meshwork tissue and into a portion of Schlemm's Canal and the shaft is configured to be advanced along a circumferential contour of Schlemm's Canal away from the portion of Schlemm's Canal. The first tissue disruptor is configured to disrupt trabecular meshwork tissue as the shaft advances along the circumferential contour of Schlemm's Canal. The second tissue disruptor is configured to disrupt tissue upon retraction of the shaft and not as the shaft is advanced along the circumferential contour.

P Embodiment 2. The device of P Embodiment 1, wherein the distal-most end is an atraumatic tip.

P Embodiment 3. The device of P Embodiment 2, wherein the atraumatic tip is configured for circumferential gonio-traction.

P Embodiment 4. The device of P Embodiments 2 or 3, wherein the atraumatic tip on the shaft is located 1 mm-3 mm away from the first tissue disruptor and the second tissue disruptor.

P Embodiment 5. The device of P Embodiments 1-4, wherein the shaft is a tube having a lumen extending along a longitudinal axis and defined by a cylindrical wall.

P Embodiment 6. The device of P Embodiment 5, wherein the first tissue disruptor is a segment of the cylindrical wall of the tube projecting inward at an angle relative to the longitudinal axis.

P Embodiment 7. The device of P Embodiments 5 or 6, wherein the second tissue disruptor is a discontinuity in the cylindrical wall of the tube.

P Embodiment 8. The device of P Embodiment 7, wherein the discontinuity forms a window having a leading edge facing proximally and a trailing edge facing distally.

P Embodiment 9. The device of P Embodiment 8, wherein the trailing edge is blunt and does not disrupt tissue during advancement of the shaft, and wherein the leading edge is sharpened and disrupts tissue during retraction of the shaft.

P Embodiment 10. The device of P Embodiments 1-9, further comprising an inner member comprising a control wire and an atraumatic distal tip, the inner member movable through the lumen of the tube.

P Embodiment 11. The device of P Embodiment 10, wherein the atraumatic distal tip is configured to be positioned distal to the distal-most end of the shaft.

P Embodiment 12. The device of P Embodiments 1-11, wherein the radially inward surface of the curve of the distal end region is connected to the radially outward surface by two lateral sides.

P Embodiment 13. The device of P Embodiment 12, wherein the first tissue disruptor has a distal face, a proximal face, and a maximum thickness, the distal face projecting a distance from a first thickness of the shaft distal to the first tissue disruptor forming the maximum thickness and the proximal face tapering down from the maximum thickness to a second thickness of the shaft proximal to the tissue disruptor, and wherein the first tissue disruptor is a blunt tissue-engaging surface without any cutting element.

P Embodiment 14. The device of P Embodiment 13, wherein the first thickness of the shaft between the inward and outward surfaces proximal to the disruptor is 100-150 microns and the second thickness of the shaft between the inward and outward surfaces distal to the disruptor is 100-150 microns.

P Embodiment 15. The device of P Embodiment 14, wherein the maximum thickness of the tissue disruptor between the inward and the outward surfaces is about 250-600 microns.

P Embodiment 16. The device of P Embodiment 13, wherein the first thickness of the shaft between the inward and outward surfaces proximal to the disruptor is 100-2000 microns and the second thickness of the shaft between the inward and outward surfaces distal to the disruptor is 100-550 microns.

P Embodiment 17. The device of P Embodiment 16, wherein the maximum thickness of the tissue disruptor between the inward and outward surfaces is about 450-600 microns.

P Embodiment 18. The device of P Embodiments 13-17, wherein the shaft has a cross-sectional shape taken transverse to a length of the shaft between that is non-circular.

P Embodiment 19. The device of P Embodiment 18, wherein the cross-sectional shape is square or rectangular.

P Embodiment 20. The device of P Embodiments 13-19, wherein the super-elastic memory-shape material is Nitinol.

P Embodiment 21. The device of P Embodiment 20, wherein the shaft is cut from a flat sheet of Nitinol having a thickness of about 75-550 microns to form a profile of the first and second tissue disruptors.

P Embodiment 22. The device of P Embodiments 12-21, wherein the second tissue disruptor comprises one or more tines having a leading surface facing distally and a trailing surface facing proximally.

P Embodiment 23. The device of P Embodiment 22, wherein the leading surface facing distally is smooth to slide along an outer wall of Schlemm's Canal during advancement without causing tissue disruption.

P Embodiment 24. The device of P Embodiment 23, wherein the trailing surface facing proximally is sharp to catch on the outer wall of Schlemm's Canal during retraction causing tissue disruption.

P Embodiment 25. The device of P Embodiments 1-24, further comprising a proximal portion that is configured to remain outside the eye when the distal portion is inserted inside the eye.

P Embodiment 26. The device of P Embodiment 25, wherein the proximal portion comprises an actuator operatively coupled to the shaft, the actuator configured to advance the shaft distally.

P Embodiment 27. The device of P Embodiments 1-26, wherein the curve of the distal end region of the shaft has a radial curvature of 5-20 mm.

P Embodiment 28. The device of P Embodiments 1-27, further comprising a proximal housing having an introducer tube projecting from a distal end region of the housing, at least a portion of the shaft extending through a lumen of the introducer tube.

P Embodiment 29. The device of P Embodiment 28, wherein the shaft is configured to be advanced from the introducer tube.

P Embodiment 30. The device of P Embodiment 29, wherein the shaft develops a spring-load as the shaft extends from the introducer tube.

P Embodiment 31. The device of P Embodiments 29 or 30, wherein the shaft applies a radially outward force as the shaft extends from the introducer tube.

P Embodiment 32. The device of P Embodiments 29 or 30, wherein a stiffness of the shaft is varied by changing a length of the shaft extending from the introducer tube.

P Embodiment 33. The device of P Embodiments 28-32, wherein the introducer tube is a substantially rigid tube having a proximal end region that extends away from the proximal housing along a longitudinal axis and a distal end region that curves relative to the longitudinal axis.

P Embodiment 34. A device for disrupting tissue in an eye, the device comprising: a distal portion sized and configured for ab interno insertion into an anterior chamber of the eye, the distal portion comprising: an elongate, flexible shaft of super-elastic memory-shape material comprising: a distal end region shaped into a curve having a central plane, the distal end region of the shaft having a radially inward surface, a radially outward surface, and a first thickness between the radially inward surface and the radially outward surface; a probe tip at a distal-most end of the distal end region, the probe tip having a maximum thickness between the radially inward surface of the distal end region and the radially outward surface of the distal end region; a tissue disruptor proximal of the probe tip projecting away from the radially inward surface; and a neck region proximal of the probe tip and distal to the tissue disruptor, wherein the neck region has a second thickness between the radially inward surface and the radially outward surface; and wherein, during use, the distal-most end is configured to be inserted through trabecular meshwork tissue and into a portion of Schlemm's Canal and the shaft is configured to be advanced along a circumferential contour of Schlemm's Canal away from the portion of Schlemm's Canal, wherein the tissue disruptor is configured to disrupt trabecular meshwork tissue as the shaft advances along the circumferential contour of Schlemm's Canal, and wherein maximum thickness of the probe tip is greater than the first thickness of the distal end region of the shaft, and the second thickness of the neck region is less than the first thickness.

P Embodiment 35. The device of P Embodiment 34, wherein the probe tip is located 1 mm-3 mm away from the tissue disruptor.

P Embodiment 36. The device of P Embodiments 34 or 35, wherein the radially inward surface of the curve of the distal end region is connected to the radially outward surface by two lateral sides.

P Embodiment 37. The device of P Embodiments 34-36, wherein the super-elastic memory-shape material is Nitinol.

P Embodiment 38. The device of P Embodiments 34-37, wherein the curve of the distal end region of the shaft has a radial curvature of 5-20 mm.

P Embodiment 39. The device of P Embodiments 34-38, wherein the first thickness of the distal end region of the shaft is at least 120 microns up to about 150 microns.

P Embodiment 40. The device of P Embodiment 39, wherein the maximum thickness of the probe tip is greater than 180 microns up to about 360 microns.

P Embodiment 41. The device of P Embodiment 40, wherein the second thickness of the neck region is less than about 100 microns down to about 60 microns.

P Embodiment 42. A method of using the device of P Embodiments 34-41, the method comprising performing ab interno continuous goniotomy and an inner wall trabeculotomy along a segment of a circumference of an eye.

P Embodiment 43. The method of P Embodiment 42, wherein the segment is greater than 90 degrees up to about 180 degrees.

P Embodiment 44. The method of P Embodiments 42-43, further comprising positioning at least one implant within a ciliary cleft.

P Embodiment 45. The method of P Embodiment 44, wherein the positioning is performed after the continuous goniotomy and an inner wall trabeculotomy.

P Embodiment 46. The method of P Embodiments 44-45, wherein the implant is minimally-modified biological tissue.

P Embodiment 47. The method of P Embodiment 46, wherein the biological tissue is scleral, corneal, or amniotic membrane tissue.

1. A device for disrupting tissue in an eye, the device comprising:
a distal portion sized and configured for ab interno insertion into an anterior chamber of the eye, the distal portion comprising:
an elongate, flexible shaft comprising:
a distal end region;
a distal-most end;
a first tissue disruptor proximal of the distal-most end formed on an inward surface of the distal end region; and
a second tissue disruptor proximal of the distal-most end formed on an outward surface of the distal end region,
wherein, during use, the distal-most end is configured to be inserted through trabecular meshwork tissue and into a portion of Schlemm's Canal and the shaft is configured to be advanced along a circumferential contour of Schlemm's Canal away from the portion of Schlemm's Canal,
wherein the first tissue disruptor is configured to disrupt trabecular meshwork tissue as the shaft advances along the circumferential contour of Schlemm's Canal, and
wherein the second tissue disruptor is configured to disrupt tissue upon retraction of the shaft and not as the shaft is advanced along the circumferential contour.

2. The device of claim 1, wherein the distal-most end is an atraumatic tip.

3. The device of claim 2, wherein the atraumatic tip is configured for circumferential gonio-traction.

4.-11. (canceled)

12. The device of claim 1, wherein the radially inward surface of the distal end region is connected to the radially outward surface by two lateral sides.

13. The device of claim 12, wherein the first tissue disruptor has a distal face, a proximal face, and a maximum thickness, the distal face projecting a distance from a first thickness of the shaft distal to the first tissue disruptor forming the maximum thickness and the proximal face tapering down from the maximum thickness to a second thickness of the shaft proximal to the tissue disruptor, and wherein the first tissue disruptor is a blunt tissue-engaging surface without any cutting element.

14. The device of claim 13, wherein the first thickness of the shaft between the inward and outward surfaces proximal to the disruptor is 100-150 microns and the second thickness of the shaft between the inward and outward surfaces distal to the disruptor is 100-150 microns.

15. The device of claim 14, wherein the maximum thickness of the tissue disruptor between the inward and the outward surfaces is about 250-600 microns.

16. The device of claim 13, wherein the first thickness of the shaft between the inward and outward surfaces proximal to the disruptor is 100-2000 microns and the second thickness of the shaft between the inward and outward surfaces distal to the disruptor is 100-550 microns.

17. The device of claim 16, wherein the maximum thickness of the tissue disruptor between the inward and outward surfaces is about 450-600 microns.

18. The device of claim 13, wherein the shaft has a cross-sectional shape taken transverse to a length of the shaft between that is non-circular, and wherein the cross-sectional shape is square or rectangular.

19. (canceled)

20. The device of claim 1, wherein the shaft is formed of super-elastic memory-shape material.

21. (canceled)

22. The device of claim 20, wherein the shaft is cut from a flat sheet of material having a thickness of about 75-550 microns to form a profile of the first and second tissue disruptors.

23. (canceled)

24. The device of claim 12, wherein the second tissue disruptor comprises one or more tines having a leading surface facing distally and a trailing surface facing proximally.

25. The device of claim 24, wherein the leading surface facing distally is smooth to slide along an outer wall of Schlemm's Canal during advancement without causing tissue disruption and wherein the trailing surface facing proximally is sharp to catch on the outer wall of Schlemm's Canal during retraction causing tissue disruption.

26.-36. (canceled)

37. A device for disrupting tissue in an eye, the device comprising:
a distal portion sized and configured for ab interno insertion into an anterior chamber of the eye, the distal portion comprising:
an elongate, flexible shaft comprising:
a distal end region having an inward surface, an outward surface, and a first thickness between the inward surface and the outward surface;
a probe tip at a distal-most end of the distal end region, the probe tip having a maximum thickness between the radially inward surface of the distal end region and the radially outward surface of the distal end region;
a tissue disruptor proximal of the probe tip projecting away from the radially inward surface; and
a neck region proximal of the probe tip and distal to the tissue disruptor, wherein the neck region has a second thickness between the radially inward surface and the radially outward surface; and
wherein, during use, the distal-most end is configured to be inserted through trabecular meshwork tissue and into a portion of Schlemm's Canal and the shaft is configured to be advanced along a circumferential contour of Schlemm's Canal away from the portion of Schlemm's Canal, wherein the tissue disruptor is configured to disrupt trabecular meshwork tissue as the shaft advances along the circumferential contour of Schlemm's Canal, and
wherein maximum thickness of the probe tip is greater than the first thickness of the distal end region of the shaft, and the second thickness of the neck region is less than the first thickness.

38. The device of claim 37, wherein the probe tip is located 1 mm-3 mm away from the tissue disruptor.

39. The device of claim 37, wherein the radially inward surface of the distal end region is connected to the radially outward surface by two lateral sides.

40. The device of claim 37, wherein the shaft is formed of a super-elastic memory-shape material.

41. The device of claim 40, wherein the super-elastic memory-shape material is Nitinol.

42. The device of claim 37, wherein the distal end region is shaped into a curve having a central plane.

43. The device of claim 42, wherein the curve of the distal end region of the shaft has a radial curvature of 5-20 mm.

44. The device of claim 37, wherein the first thickness of the distal end region of the shaft is at least 120 microns up to about 150 microns.

45. The device of claim 44, wherein the maximum thickness of the probe tip is greater than 180 microns up to about 360 microns.

46. The device of claim 45, wherein the second thickness of the neck region is less than about 100 microns down to about 60 microns.

47.-108. (canceled)
